(19) United States
(12) Patent Application Publication
(10) Pub. No.: US 2005/0255113 A1
(43) Pub. Date: Nov. 17, 2005

(54) Methods and compositions for inhibiting polypeptide accumulation associated with neurological disorders

(75) Inventors: James S. Huston, Chestnut Hill, MA (US); Anne Messer, Slingerlands, NY (US); Jean-Michel Lecerf, Cambridge, MA (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: New York State Department of Health and Abgenix, Inc.

(21) Appl. No.: 10/952,535

(22) Filed: Sep. 27, 2004

Related U.S. Application Data
(63) Continuation of application No. 09/620,955, filed on Jul. 21, 2000, now abandoned.
(60) Provisional application No. 60/146,047, filed on Jul. 27, 1999.

(51) Int. Cl. A61K 39/395; A61K 38/17

(52) U.S. Cl. 424/145.1; 514/2

(57) ABSTRACT

The invention provides methods for inhibiting the formation of undesired intracellular polypeptide complexes or aggregates associated with neurological disorders using an intrabody.

### RELATED INFORMATION

This application claims priority to U.S. provisional application No. 60/146,047, entitled “METHODS AND COMPOSITIONS FOR INHIBITING POLYPEPTIDE ACCUMULATION ASSOCIATED WITH NEUROLOGICAL DISORDERS,” filed Jul. 27, 1999, incorporated herein in its entirety by this reference. The contents of all patents, patent applications, and references cited throughout this specification are hereby incorporated by reference in their entireties.

### GOVERNMENT SPONSORED RESEARCH

This work was supported, in part, by grants from the United States Department of Health and Human Services (No.: NS38002) and Hereditary Disease Foundation.

### BACKGROUND OF THE INVENTION

Neurodegenerative disorders are some of the most feared illnesses to strike humankind. For example, it is estimated that one out of every ten people over the age of 65 will be affected by Alzheimer's disease, a progressively debilitating illness that results in memory loss and, ultimately, death. Indeed, currently, over 4 million people in North America suffer from this disease and current treatments are essentially palliative. Similarly, Parkinson's disease affects nearly 1 million people in North America and currently there are no treatments available to postpone the onset of illness or substantially slow the progress of the disease. Another insidious neurological disorder is Huntington's disease (HD), which currently affects over 30,000 people in North America. Less common, although equally debilitating, are the prion diseases, such as new variant Creutzfeldt-Jakob disease, which also results in mental deterioration and eventually, death. A number of other neurodegenerative diseases such as frontotemporal dementia (FTD), amyotrophic lateral sclerosis (ALS), spinal and bulbar muscular atrophy (SBMA or Kennedy disease), dentatorubral-pallidoluysian atrophy (DRPLA), and the spinocerebellar ataxias (e.g., SCA 1-SCA 7) also exhibit similar courses. The etiology of many of these diseases is still incompletely understood, although it appears that most of these diseases may result from the production of an altered polypeptide in cells of the nervous system (see, e.g., Hurtley, S., Science 282:1071 (1998); Shoulson, I., Science 282:1072-1074 (1998); Hardy et al., Science 282:1075-1079 (1998); and Price et al., Science 282:1079-1083 (1998); Klement et al., Cell 95:41-53 (1998)).

For example, Huntington's disease is associated with selective neuronal cell death occurring primarily in the cortex and striatum (Harper, Huntington's Disease, Edition 22, Saunders Co., Ltd. (1991); Vonsattel et al., J. Neuropath. Exp. Neurol. 44:559-577, (1985)). The disorder appears to be caused by an expanded CAG repeat in the first exon of the ht gene encoding a polyglutamine expansion in the huntingtin polypeptide; this is a large ˜350 kDa polypeptide of unknown function found ubiquitously in human cells, but at its highest concentrations in cells of the cortex and striatum (Huntington's Disease Collaborative Research Group, Cell 72:971-983 (1993)). The CAG repeat is highly polymorphic and varies from 6 to 39 repeats on chromosomes of unaffected individuals and from 36 to 180 repeats on chromosomes of HD patients (Rubinsztein et al., Am. J Hum. Genet. 59:16-22 (1996); Sathasivam et al., Hum. Genet. 99:692-695 (1997)). The majority of adult onset cases have expansions ranging from 40 to 55 units, whereas expansions of 70 and above invariably cause the juvenile form of the disease. Within the brain, the huntingtin polypeptide has been found predominantly in neurons and is primarily a cytosolic polypeptide, a fraction of which is associated with vesicles and/or microtubules, suggesting that it may play a functional role in cytoskeletal anchoring or transport of vesicles (DiFiglia et al., Neuron 14:1075-1081 (1995); Gutekunst et al., P.N.A.S. 92:8710-8714 (1995); Sharp et al., Neuron 14:1065-1074 (1995)). Huntingtin has also been detected in the nucleus (Hoogeveen et al., Hum. Mol. Genet. 2:2069-2073 (1993); de Rooij et al., Hum. Mol. Genet. 5:1093-1098 (1996)), suggesting that transcriptional regulation cannot be ruled out as a possible function of this polypeptide. Huntingtin is also found in cells of the pancreas, where the presence of pathological expanded repeats in huntingtin is associated with the onset of diabetes associated with Huntington's disease (Hurlbert et al., Diabetes 48:649-651 (1999); Farrer, L. Clin. Genet. 27:62-67 (1985)).

In addition to HD, CAG/polyglutamine expansions have been found in at least six other inherited neurodegenerative disorders including spinal and bulbar muscular atrophy, dentatorubral-pallidoluysian atrophy, and the spinocerebellar ataxia types 1, 2, 3, and 6 (Bates et al., Human. Mol. Genet. 6:1633-1637 (1997); Trottier et al., Nature 378:403-406 (1995)). The normal and expanded size ranges are comparable with the exception of SCA6 in which the expanded alleles are smaller and the mutation is likely to act by a different route. However, in all cases the CAG repeat is located within the coding region and is translated into a stretch of polyglutamine residues. Although the polypeptides harboring the polyglutamine sequences are unrelated and mostly of unknown function, it is likely that the mutations act through a similar mechanism (Sisodia, S., Cell 95:1-4 (1998)). These polypeptides are usually widely expressed and generally localized in both the nucleus and cytoplasm. In addition, neurodegenerative disease such as Alzheimer's disease, Parkinson's disease, Huntington's disease, prion disease, frontotemporal dementia, amyotrophic lateral sclerosis, spinal and bulbar muscular atrophy, dentatorubral-pallidoluysian atrophy, spinocerebellar ataxia type 1, spinocerebellar ataxia type 2, spinocerebellar ataxia type 3, spinocerebellar ataxia type 4, spinocerebellar ataxia type 5, spinocerebellar ataxia type 6, and spinocerebellar ataxia type 7 have also been associated with the production of an altered polypeptide in cells of the nervous system (Hardy et al., Science 282:1075-1079 (1998)).

Accordingly, it has been speculated that the accumulation of altered polypeptides in cells of the nervous system may presage the neuronal degeneration of a subset of cells and the onset of disease.

### SUMMARY OF THE INVENTION

Methods for managing the debilitating effects of neurological disorders involving the accumulation of intracellular polypeptides in abnormal multimers or aggregates lies in preventing the formation of these complexes or aggregates before they result in a pathology.

To this end, novel compositions and methods have been developed that inhibit the formation of these neurodegenerative polypeptides and their complexes or aggregates.

The present invention is based on the discovery that contacting intracellular, pathological huntingtin polypeptide with an intrabody can prevent a hallmark of its abnormal pathology, the formation of polypeptide aggregates. Moreover, such intrabodies can both inhibit the formation of the aggregates in a manner that allows normal polypeptide breakdown within cells, and, when linked to a targeting signal, can also retarget the unwanted polypeptide for destruction in, e.g., a lysosome or a proteasome.

Accordingly, in one aspect, the invention provides a method for inhibiting the formation of intracellular aggregates of selected polypeptides including, the step of contacting a polypeptide capable of forming complexes with a polypeptide binding molecule, e.g., an intrabody that specifically binds to the polypeptide in a manner to minimize aggregation, thereby reducing the formation of intracellular aggregates.

In a second aspect, the present invention provides a method for inhibiting the formation of intracellular aggregates of selected polypeptides in a subject including, administering to a subject at risk of having intracellular aggregates, a polypeptide binding molecule, e.g., an intrabody, which specifically binds to the polypeptide in a manner to minimize aggregation thereby reducing the intracellular aggregates. In one embodiment, the subject is at risk for a neurological disorder, and preferably, is a human patient. In another embodiment, the subject is an experimental animal, and preferably, a Huntington's disease animal model.

In a third aspect, the present invention provides a method for treating a subject having, or likely to have, a neurological disorder including, administering to the subject a polypeptide binding molecule, e.g., an intrabody which specifically bind to a polypeptide capable of forming polypeptide aggregates or complexes associated with a neurological disorder, thereby inhibiting the aggregates or complexes from forming.

In a fourth aspect, the invention provides a method for identifying a polypeptide binding molecule, e.g., an intrabody, or a functional fragment thereof, which specifically recognizes a polypeptide capable of forming intracellular polypeptide aggregates or complexes comprising, providing a polypeptide capable of forming intracellular polypeptide aggregates; contacting the polypeptide with a test polypeptide binding molecule, e.g., an intrabody or functional fragment thereof; and determining the ability of the test polypeptide binding molecule, e.g., an intrabody or functional fragment thereof, to specifically recognize the polypeptide, thereby identifying a polypeptide binding molecule, e.g., an intrabody or a functional fragment thereof, which specifically recognizes a polypeptide capable of forming intracellular polypeptide aggregates.

In one embodiment of the above aspect, the intrabody of the invention may be multivalent, have a spacer region, and may be selected from an expression library, such as a phage display library, and include, for example, Fv sequences.

In a preferred embodiment, the intrabody has a binding affinity (association constant, Ka) for a given polypeptide of at least 10⁵ M⁻¹, more preferably at least 10⁶ M⁻¹, whereas if the intrabody is multivalent, each valence may have a binding affinity for the polypeptide of at least 10² M⁻¹, and preferably, 10³ M⁻¹. In one embodiment, the selected intrabody has a greater affinity for a mutant polypeptide as compared to wild type polypeptide. In another embodiment, the selected intrabody has a specific affinity for the wild type polypeptide, e.g., a region of the polypeptide common to both the wild type polypeptide and mutant polypeptide.

In a fifth aspect, the invention provides a method for identifying a compound which specifically recognizes a polypeptide capable of forming undesired intracellular polypeptide aggregates or complexes including, providing a polypeptide capable of forming intracellular polypeptide aggregates; providing a test polypeptide binding molecule, e.g., an intrabody or functional fragment thereof, that binds the polypeptide; incubating the polypeptide and intrabody fragment or fragment thereof with a binding molecule; and determining the ability of the test compound to alter the binding of the polypeptide binding molecule, e.g., an intrabody or functional fragment thereof, wherein those binding molecules that bind the intrabody are eliminated, thereby identifying the test compound as capable of interacting with a polypeptide capable of forming intracellular polypeptide aggregates. In a preferred embodiment, the method is applied, and can be reapplied, to a variegated library of at least 10², 10³, 10⁴, 10⁵, 10⁶, 10⁷, 10⁸, and, preferably, 10⁹ or more different binding molecules.

In a related embodiment, the test compound is a small organic molecule, peptide, or natural product extract.

In a sixth aspect, the invention provides an isolated nucleic acid molecule encoding an intrabody, or functional fragment thereof, which binds to a selected polypeptide capable of forming intracellular polypeptide aggregates associated with a neurological disorder.

In one embodiment, the invention provides the nucleic acid sequence set forth in SEQ ID NO: 1 or SEQ ID NO: 3, and preferably, a nucleic acid sequence set forth in SEQ ID NO: 5 that, respectively, encode the amino acid sequence set forth in SEQ ID NO: 2, SEQ ID NO: 4, and SEQ ID NO: 6 and these sequences correspond to a preferred intrabody, or fragment thereof.

In another embodiment, the intrabody of the invention is encoded by a nucleic acid molecule that hybridizes to the nucleic acid molecule of SEQ ID NO: 1, SEQ ID NO: 3, or SEQ ID NO: 5 under stringent conditions. In a preferred embodiment, the nucleic acid is derived from a mammal. Such a nucleic acid may also be encoded by a vector, preferably an expression vector, such as, for example, an expression vector derived from a virus, e.g., adenovirus, adeno-associated virus, a retrovirus, or herpes simplex virus. Moreover, the vector encoding the intrabody or the polypeptide intrabody may be in the form of a pharmaceutical composition that includes a pharmaceutically acceptable carrier.

In even another embodiment, the invention provides a host cell that comprises any of the aforementioned vectors, the host cell being preferably of neuronal origin or part of a tissue of neuronal origin.

In a seventh aspect, the invention provides a polypeptide based polypeptide binding molecule, preferably an intrabody, or functional fragment thereof, which binds to a selected polypeptide capable of forming intracellular polypeptide aggregates associated with a pathology, e.g., a neurological disorder.

In an eighth aspect, the invention provides a method for inhibiting the formation of intracellular aggregates of a selected polypeptide in an animal by immunizing the animal, e.g., a human patient, with an immunogen having an epitope in common with the selected polypeptide, where the immunizing provokes a host antibody immune response sufficient for inhibiting the formation of aggregates, e.g., intracellular aggregates of the selected polypeptide from occurring. In a preferred embodiment, the immunogen is an expressible nucleic acid vaccine, e.g., a DNA vaccine, encoding a polypeptide comprising an epitope in common with a polypeptide such as, e.g., Amyloid Precursor Protein, Presenilin 1, Presenilin 2, α-2 Macroglobulin, Apolipoprotein, α-Synuclein, huntingtin, Prion protein, Tau, SOD, AR, Atrophin 1, Ataxin 1, Ataxin 2, Ataxin 3, CACNL1A4, SCA7, and preferably, Huntington.

In a related embodiment, the animal has, or is at risk for having, Huntington's disease or Huntington's associated diabetes (characterized by, e.g., high fasting blood glucose levels and/or low insulin levels).

In still another embodiment, the invention provides a transgenic animal engineered to express a nucleic acid encoding an intrabody where, preferably, the intrabody can selectively bind to a polypeptide capable of forming aggregates associated with a neurological disorder, for example Huntington's disease caused by huntingtin polypeptide, e.g., a huntingtin polypeptide having additional glutamine residues as compared to a corresponding wild type huntingtin polypeptide.

In each of the foregoing aspects, the invention provides in a preferred embodiment a method for inhibiting the formation of an intracellular aggregates involving a polypeptide such as Amyloid Precursor Protein, Presenilin 1, Presenilin 2, α-2 Macroglobulin, Apolipoprotein, α-Synuclein, huntingtin, Prion protein, SOD, AR, Atrophin 1, Ataxin 1, Ataxin 2, Ataxin 3, CACNL1A4, or SCA7 and preferably, either huntingtin, Tau, or any mutant polypeptide thereof including any fragment of a wild type polypeptide or mutant polypeptide.

In another embodiment of the foregoing aspects, the polypeptide includes a naturally-occurring polypeptide having additional glutamine residues as compared to a corresponding wild type polypeptide where, preferably, the polypeptide is huntingtin, more preferably, huntingtin polypeptide having additional glutamine residues as compared to a corresponding wild type huntingtin polypeptide.

In even another embodiment of the foregoing aspects, the intrabody of the invention may be multivalent or have a spacer region between binding sites having the same or distinct specificities.

In yet another embodiment of the foregoing aspects, the intrabody of the invention may include a small molecule peptide, peptidomimetic antibody, an antibody fragment, or preferably, an intrabody, e.g., an intrabody containing an amino acid sequence corresponding to a targeting signal, e.g., ubiquitin thereby allowing the intrabody to retarget the target polypeptide to a proteasome.

In still another embodiment of the foregoing aspects, the polypeptide binding molecule of the invention may include an amino acid sequence corresponding to a targeting signal thereby allowing the intrabody to retarget the target polypeptide to a particular cellular location. In a preferred embodiment, the targeting signal is cytoplasmic, nuclear, lysosomal, plasma membrane-associated, endoplasmic reticulum-associated, peroxisomal, or proteosomal and preferably, nuclear or lysosomal.

In yet another embodiment of the foregoing aspects, the intrabody includes the a minimal sFv binding region specific for huntingtin as provided in SEQ ID NO: 2 or SEQ ID NO: 4 and preferably, the amino acid sequence of the intrabody α-Nt-HD-C4 sFv provided in SEQ ID NO: 6. In another embodiment, the polypeptide may be contacted with the intrabody in vivo or in vitro.

Accordingly, it will be appreciated that the invention comprises a number of advantages. For example, the intrabody may be administered in the form of an expressible gene where it can work intracellularly and confer protection to the cell at an intracellular level. In addition, the compositions and methods of the invention are of a design such that they can function independently of the immune system and are unlikely to trigger any undesired immune response.

Other features and advantages of the invention will be apparent from the following detailed description, and from the claims.

### BRIEF DESCRIPTION OF THE DRAWINGS

FIG. 1 shows the wild type N-terminal amino acid sequence of several polypeptides that are polyglutamine-rich and are associated with neurological disease when mutated to pathological lengths of polyglutamine (i.e., poly Q). For comparison, a TBP polypeptide having a polyglutamine stretch that is not associated with neurological disease is shown. The peptide used for raising the huntingtin-specific C4 sFv intrabody is also shown.

FIG. 2 shows the amino acid sequence of a number of targeting signals that can target heterologous polypeptides to a particular cellular locale. The hemagglutinin (HA) tag is a peptide that is tightly bound by appropriate antibodies for immunostaining used to visualize the intrabody separately from the antigen.

FIGS. 3A-D show, using fluorescence microscopy, the length-dependent aggregation of model huntingtin-GFP fusion polypeptides (i.e., HD-25Q-GFP, HD-47Q-GFP, HD-72Q-GFP, HD-104Q-GFP) having increasing numbers (i.e., 25, 47, 72, 104) of glutamine residues. When expressed in cells (COS-7, in this figure), model huntingtin-GFP fusion proteins having, respectively, 72 and 104 glutamine residues (HD-72Q, HD-104Q), readily aggregate into small brilliantly fluorescent foci (FIGS. 3C-D). In contrast, model huntingtin-GFP polypeptides having only a wild type number (25) of glutamine residues (HD-25Q) remain disaggregated and show only diffuse cytoplasmic GFP fluorescence, or slightly pathological polypeptide in terms of polyQ length (HD-47Q), one sees both a tendency to form aggregates but many cells maintain the antigen evenly distributed throughout the cell (FIGS. 3A-B).

FIGS. 4A-B show, using fluorescence microscopy, the ability of model huntingtin-GFP fusion polypeptides having 72 and 104 glutamine residues (pHD-72Q, FIG. 3B; pHD-104Q, FIG. 3A) to aggregate into small intensely staining areas when expressed in cerebellar slices derived from mice and biolistically transfected using a gene gun.

FIGS. 5A-H show the ability of an intrabody to specifically bind and retarget a model huntingtin polypeptide in a cell. In FIGS. 5A and 5B, the distribution of cells using phase contrast light microscopy is shown. FIGS. 5C, 5E, and 5G, using fluorescence microscopy, show that cells coexpressing a model huntingtin-GFP fusion polypeptide (HD-25Q-GFP) (FIG. 5E) and an intrabody that specifically binds the model huntingtin polypeptide and comprises a nuclear targeting signal (αNt-HD-C4 sFV-NLS) can retarget the distribution of the model huntingtin polypeptide to the nucleus as demonstrated by a confluence of staining in the nucleus (FIG. 5G). In contrast, cells expressing an intrabody that binds an unrelated polypeptide fail to retarget the distribution of the model huntingtin polypeptide (FIGS. 5D, 5F, and FH).

FIGS. 6A-G show that the ability of an intrabody to retarget the localization of a particular polypeptide is a function of the binding specificity of the intrabody. For example, in FIGS. 6C-G, cells (COS-7) coexpressing the glutamine rich DRPLA polypeptide (GFP-DRPLA-35Q) and an intrabody against either a model huntingtin polypeptide (H-HD-C4 sFv; FIGS. 6D, 6F) or another unrelated polypeptide (Negative Control sFv; FIGS. 6C, 6E, and 6G) show no change in the cellular distribution of DRPLA polypeptide. FIGS. 6A and 6B show the overall distribution of cells using phase contrast light microscopy.

FIGS. 7A-H show, using fluorescence microscopy, that an intrabody can specifically retarget the cellular distribution of a mutant model huntingtin polypeptide (i.e., having 104 glutamine repeats) to the nucleus (FIGS. 7C, 7E, and 7F) whereas an irrelevant intrabody does not (FIGS. 7B, 7D, and 7G). In FIGS. 7A and 7B, the distribution of cells using phase contrast light microscopy is shown.

FIGS. 8A-G show, using fluorescence microscopy, that the formation of intracellular polypeptide aggregates was inhibited by intrabody expression. Two different polypeptides representing huntingtin polypeptide with mutations known to be associated with neurological disease in humans were demonstrated to form intracellular aggregates when expressed in cells (FIGS. 8A and 8E). The formation of these aggregates was inhibited by three different intrabodies having either no targeting signal (see FIGS. 8B, 8F), a nuclear targeting signal (see FIGS. 8C, 8G), or a lysosomal targeting signal (FIGS. 8D, 8G). FIGS. 8C-D, 8F-G also show that an intrabody with a targeting signal (either nuclear or lysosomal), in addition to reducing intracellular polypeptide aggregation, also caused retargeting of the model huntingtin polypeptide.

FIG. 9 shows a photograph of a Coomassie stained electrophoretic gel demonstrating the presence and the apparent molecular weight of various polypeptides used in demonstrating the binding specificity of the model huntingtin-specific C4 sFv intrabody (as probed by immunoblot in FIG. 10).

FIG. 10 shows an immunoblot demonstrating that only a huntingtin polypeptide epitope (lanes 3-4, 6-7) can be used to affinity purify the model huntingtin polypeptide-specific C4 sFv intrabody whereas the unrelated DRPLA polypeptide (Lanes 2 and 5) fails to interact with the C4 sFv intrabody.

FIG. 11A shows results of direct binding studies of the anti-huntingtin intrabody C4 sFv to immobilized GST-fusion proteins when analyzed by ELISA.

FIG. 11B shows results of kinetic binding affinity studies of the anti-huntingtin intrabody C4 sFv when analyzed by BIAcore. A range of concentration (60 to 100 nM) of anti-huntingtin intrabody C4 sFv was used to measure the association rate (kₒₙ) on 50 RU of biotinylated-HD peptide bound to a streptavidin sensor chip.

FIG. 12 shows retargeting of GFP-fusion HD fragments to the nucleus by anti-huntingtin intrabody Nt-HD C4 sFv-NLS. Panels B, D, and F show immunofluorescence of HD-Q104-Myc-HIS₆ and C4 sFv-NLS or negative sFv-NLS intrabodies (Panels A, C, and E), after 48 h co-transfection in COS-7. HD-Q104-Myc-HIS₆ was visualized by immunostaining with the anti-myc 9E10 MAb, followed by FITC-labeled goat anti-mouse IgG antibodies (Panels A, B). The intrabodies were immunostained with polyclonal anti-HA antibodies, followed by rhodamine-labeled goat anti-rabbit IgG antibodies (Panels C, D; Panels E and F represent dual staining of the same fields).

FIG. 13 shows relocalization of GFP-HD-Q104 (b, d, f; 40×) or GFP-DRPLA-Q81 (a, c, e; as negative control; 20×) in COS-7 cells stably expressing the anti-huntingtin intrabody C4 sFv-NLS. Panels A and B show GFP expression, panels C and D show rhodamine staining of C4 sFv-NLS intrabody, and panels E and F show dual staining of the same fields.

FIG. 14 shows anti-HD C4 sFv inhibition of pathogenic length HD-polyQ-GFP aggregation. The upper panel shows digital fluorescent microscopy images of BHK-21 cells transfected with HD-polyQ-GFP (Q=25, 72 and 104) alone or with C4 or control sFv (ML3-9) at a plasmid transfection ratio of 5:1 (sFv:HD). Cells are shown photographed live at 48 hours after transfection by the calcium phosphate method; aggregates appear as intense foci of GFP signals. Similar results were seen with HD-72Q-GFP co-transfections. The lower panel represents a quantification of aggregates following 5:1 co-transfections of sFv (C4 or ML3-9) or parent vector (pcDNA) and HD-72Q-GFP in COS-7, BHK-21 and HEK 293 cells. Cells were lysed with 2% SDS/2% Triton X-100/50 mM Tris at 48 hours after co-transfection. The number of insoluble, fluorescent aggregates in six or eight random fields was counted. Bars represent means of sextuplicate co-transfections ±SEM. C4 significantly reduced the number of aggregates when compared to control ML3-9 or pcDNA (p<0.0005). Similar results were seen with HD-104Q-GFP.

FIG. 15 shows anti-HD C4 sFv inhibition of pathogenic length HD-polyQ-GFP aggregation. The upper panel shows digital fluorescent microscopy images of double-labeled cells co-transfected with 5:1 C4:HD-polyQ-GFP HD-polyQ-GFP (green), C4 sFv (red, detected by anti-HA antibody and Alexa 568-conjugated secondary antibody), and merged image. Some aggregates are found in 104Q co-transfections (never in 25Q), often in cells not expressing C4 (arrowheads), but in some dually-transfected cells as well (arrow). The lower panel shows digital images of immunoblot analysis of HD-72Q-GFP protein expression in co-transfections. Cell lysates were collected from 5:1 co-transfections (sFv:HD-72Q-GFP) in duplicate at 24 hours (prior to prominent aggregate formation and loss of HD-72Q-GFP to the insoluble compartment) and subjected to SDS-PAGE. The membrane was probed with 1:1000 anti-AFP primary antibody (Quantum), stripped and reprobed with 1:500 anti-actin antibody (Sigma). HD-72Q-GFP bands were of similar intensity after detection by HRP-conjugated secondary antibody and chemiluminescence.

FIG. 16 shows glucose levels in mice (wild type and transgenic Huntingtin strains (HD)) treated and untreated with a DNA vaccine encoding a huntingtin epitope.

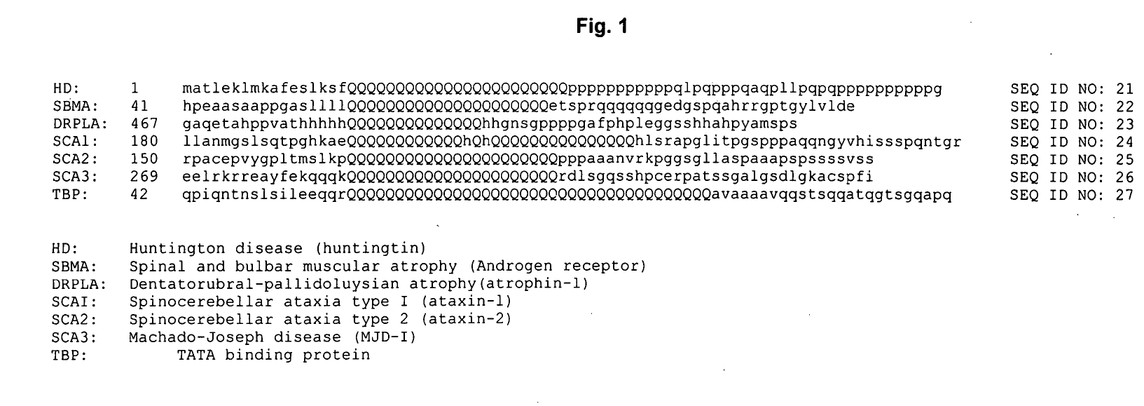

### DETAILED DESCRIPTION OF THE INVENTION

In order for the full scope of the invention to be clearly understood, the following definitions are provided.

I. Definitions

As used herein the term “inhibiting the formation” is intended to include the ability of a compound or intrabody to reduce or eliminate, e.g., reduce the rate of accumulation, lower the available polypeptide level to prevent accumulation, or otherwise offset or eliminate the accumulation or the formation of an undesired polypeptide in a cell, e.g. by maintaining the antibody-antigen complex in a soluble state that discourages aggregate formation and allows polypeptide breakdown of the HD-analogue polypeptides.

The term “polypeptide aggregates” is intended to include any undesired, aberrant, or abnormal accumulation of a polypeptide, polypeptide-polypeptide interaction, polypeptide complex, or polypeptide aggregate. The term is also intended to include polypeptide aggregates which the cell may form in an effort to minimize harm to the cell by localizing the offending polypeptide to a certain intracellular space. In a preferred embodiment, the polypeptide is an altered polypeptide associated with disease. e.g., a neurological disease. The term is intended to include, e.g., huntingtin aggregate bodies, amyloid plaques, neurofibrillary tangles, Lewy bodies, prion plaques, Lewy-like bodies, and any undesired complex or aberrant accumulation or inclusion, found in a cell, such as, e.g., a cell of neuronal origin. Still further, the term may also refer to extracellular polypeptide aggregates. The terms “polypeptide” or “polypeptides” and “protein” or “proteins” are used interchangeably throughout the specification.

The term “selected polypeptide” or “target polypeptide” is intended to include any polypeptide that forms undesired accumulations or aggregations, e.g., polypeptide aggregates. Typically, the selected or target polypeptide is an altered polypeptide when compared to a corresponding wild type polypeptide.

The term “polypeptide binding molecule” includes any molecule that is capable of specifically binding to a polypeptide capable of forming, e.g., undesirable accumulations, conformations, or aggregations. Accordingly, the term includes small molecules, peptides, peptidomimetics, antibodies (including e.g., endogenous antibodies, i.e., produced by immune system of the host animal; exogenous antibodies, i.e., antibodies administered in the form of an expressible nucleic acid/s, cell/s containing the foregoing nucleic acid/s, or as a polypeptide therapy), antibody fragments (e.g., Fab fragments), and preferably, as an intrabody, or analogous engineered protein from the immunoglobulin superfamily which, preferably, acts intracellularly to bind an antigen in a cell.

The term “intrabody” is intended to include any single-chain polypeptide binding agent that can specifically bind intracellularly to a target polypeptide. Typically, an intrabody is a single-chain Fv (sFv) that comprises a minimal light chain variable region linked to a heavy chain variable region. The intrabody is typically administered in the form of a polypeptide therapy but may also be administered in the form of a expressible nucleic acid or in form of a cell expressing such a nucleic acid.

The term “multivalent” is intended to include any agent or intrabody that comprises more than one binding site having, e.g., one or more binding specificities determined by distinct sFv components within the molecule.

The term “spacer region” is intended to include any region of a peptide-based binding agent or intrabody designed to connect one or more functional domains, e.g., one sFv region to another, or an sFv binding site to a targeting signal.

The term “linker region” is intended to comprise the peptide sequence of the sFv that connects a variable light chain region and a heavy chain region as a V_{H}-linker-V_{L} or V_{L}-linker-V_{H}, where the linker is encoded at the genetic level.

The term “retargets” is intended to include the ability of a binding agent or intrabody to, upon binding to a target polypeptide, redirect the intracellular location of the polypeptide.

The term “targeting signal” is intended to include any art recognized amino acid sequence capable of, when linked to a heterologous polypeptide, directing the intracellular transport of the polypeptide to a particular location in a cell. The term is intended to include, e.g., targeting signals that are nuclear, cytoplasmic, plasma membrane-associated, endoplasmic reticulum-associated, lysosomal, peroxisomal, or proteasomal.

The term “wild type huntingtin polypeptide” is intended to include a huntingtin polypeptide having only a normal number of glutamine residues not associated with disease. Typically, any huntingtin polypeptide having less than 38 glutamine residues is not associated with disease and may be considered a normal or wild type polypeptide.

The term “neurological disorder” is intended to include any disease or condition involving cells of the central nervous system and/or peripheral nervous system. The term is intended to include, e.g., Alzheimer's disease, Parkinson's disease, Huntington's disease, prion diseases, frontotemporal dementia (FTD), amyotrophic lateral sclerosis (ALS), spinal and bulbar muscular atrophy (SBMA or Kennedy disease), dentatorubral-pallidoluysian atrophy (DRPLA), and the spinocerebellar ataxias (e.g., SCA 1-SCA 7).

The term “expression library” is intended to include any library comprising expressible nucleic acids.

The term “phage display library” is intended to include its art recognized meaning and typically refers to any collection of expressible nucleic acids whose expression and/or propagation is facilitated by phage-packaged nucleic acid sequences. In one typical embodiment, the sFv is fused to a phage coat protein so that the sFv antibody combining site (the specific binding of which is the phenotype) is available for antigen binding on the phage surface, and its genotype (the DNA encoding the sFv) is contained within the phage antibody genetic material.

The term “Fv” is intended to include any antigen binding fragment of an antibody, such as, e.g., Fab, Fv, Fd, V_{H}, or V_{L}. The term “sFv” is the single-chain Fv, intended to include the minimal light chain variable region linked to the minimal heavy chain variable region necessary to form a binding polypeptide capable of interacting with an epitope. Typically, an intrabody comprises at least one Fab, Fd, Fv or sFv region.

The term “mutant polypeptide” is intended to include any polypeptide or representation thereof that differs from its corresponding wild type polypeptide by having at least one amino acid substitution or addition, for example a glutamine addition. Typically, a mutant polypeptide will have an amino acid substitution, and altered stretch of amino acids due to a frame shift or altered splice site, or an expansion of one or more glutamines. In other cases the “mutant polypeptide” refers to abnormal conformation relative to the wild type molecule, which results in the abnormal formation of aggregated polypeptide species.

The term “variegated library” is intended to include any collection of nucleic acids or compounds, e.g., organic molecules or natural product extracts.

The term “cell of neuronal origin” is intended to include any cell derived from a tissue of the central nervous system or peripheral nervous system. The term is intended to include, e.g., neurons (including, e.g., motor neurons, sensory neurons), Schwann cells, Purkinje cells, astrocytes, microglial cells, ependymal cells, and oligodendrocytes. The term is also intended to include any cell having neuronal cell characteristics that has been derived from, e.g., embryonic stem cells or any other totipotent or multipotent cell or tissue.

The term “tissue sample of neuronal origin” is intended to include any multicellular sample derived from a tissue of the central nervous system or peripheral nervous system. The term is intended to include for example, whole brain slices and other samples that, typically, contain one or more different cell types.

The term “proteasome” is intended to include its normal art recognized meaning. Typically, a proteasome refers to a multisubunit polypeptide moiety in a cell capable of degrading cellular proteins and thus contributing to the regulation of the life-span of many proteins and also providing a source of peptides.

The term “ubiquitin” is intended to include its normal art recognized meaning. The term is intended to include a ubiquitin amino acid sequence from any species, including human, that, when fused to a heterologous polypeptide, is suitable for targeting the heterologous polypeptide for destruction, by, e.g., a proteasome.

The term “specifically recognize” is intended to include the ability of a molecule, e.g., an intrabody or a binding molecule, to specifically bind to a selected polypeptide.

The terms “Amyloid Precursor Protein”, “Presenilin 1”, “Presenilin 2,” “α-2 Macroglobulin”, “Apolipoprotein”, “α-Synuclein”, “huntingtin”, “Prion protein”, “Tau”, “super oxide dismutase (SOD)”, “androgen receptor (AR)”, “Atrophin 1”, “Ataxin 1”, “Ataxin 2”, “Ataxin 3”, “CACNL1A4”, and “SCA7” are intended to include their normal art recognized meaning.

The terms “Alzheimer's disease”, “Parkinson's disease”, “Huntington's disease”, “prion disease”, “frontotemporal dementia (FTD)”, “amyotrophic lateral sclerosis (ALS)”, “spinal and bulbar muscular atrophy (SBMA or Kennedy disease)”, “dentatorubral-pallidoluysian atrophy (DRPLA)”, “spinocerebellar ataxia type 1 (SCA1)”, “spinocerebellar ataxia type 2 (SCA2)”, “spinocerebellar ataxia type 3 (SCA3 or Machado-Joseph disease (MJD))”, “spinocerebellar ataxia type 4 (SCA4)”, “spinocerebellar ataxia type 5 (SCA5)”, “spinocerebellar ataxia type 6 (SCA6)”, and “spinocerebellar ataxia type 7 (SCA7)” are intended to include their normal art recognized meaning.

The term “selected polypeptide” is intended to include any polypeptide that has been identified as forming undesired accumulations or polypeptide aggregates in a cell. Typically the polypeptide is associated with a disease and is altered, e.g., comprises an amino acid substitution or polyglutamine expansion as compared to the corresponding wild type polypeptide, or assumes an abnormal tertiary structure as compare to the corresponding tertiary structure of the wild type polypeptide, or both.

The term “host cell” is intended to include a cell suitable for genetic manipulation that can, e.g., incorporate heterologous polynucleotide sequences by, e.g., transfection, lipofection, or infection. The cell can be a microorganism or a higher eukaryotic cell. The term is intended to include progeny of the cell originally transfected. In preferred embodiments, the cell is a cell of neuronal origin.

The term “heterologous polynucleotide segment” is intended to include a polynucleotide segment that encodes one or more polypeptides or portions or fragments of polypeptides. A heterologous polynucleotide segment may be derived from any source, e.g., eukaryotes, prokaryotes, viruses, phage, or synthetic polynucleotide fragments.

The term “derived from” is intended to include the isolation (in whole or in part) of a polynucleotide segment from an indicated source. The term is intended to include, for example, direct cloning, PCR amplification, or artificial synthesis from, or based on, a sequence associated with the indicated polynucleotide source.

The term “transgenic animal” is intended to include an animal, e.g., a non-human mammal, e.g., a swine, a monkey, a goat, or a rodent, e.g., a mouse, in which one or more, and preferably essentially all, of the cells of the animal include a transgene. The transgene is introduced into the cell, directly or indirectly by introduction into a precursor of the cell, e.g., by microinjection, transfection or infection, e.g., by infection with a recombinant virus. The term genetic manipulation is directed to the introduction of a recombinant DNA molecule. This molecule may be integrated within a chromosome, or it may be extrachromosomally replicating DNA.

The term “administering” is intended to refer to dispensing, delivering or applying the therapeutic agent to an animal or human by any suitable route for delivery of the therapeutic agent to the desired location in the animal or human, including delivery by either the parenteral or oral route, intramuscular injection, subcutaneous/intradermal injection, intravenous injection, buccal administration, transdermal delivery, intracranial delivery, and administration by the intranasal or respiratory tract route. The term “administering” is further intended to refer to bringing the therapeutic agent into close proximity with a cell, such that the therapeutic agent can exert its effects on the cell.

The term “vector” is intended to include a nucleic acid molecule capable of transporting another nucleic acid to which it has been linked. In one embodiment, the nucleic acid linked to a vector encodes a peptide binding molecule or intrabody.

II. Polypeptide Binding Molecules

In general, the present invention relates to molecules that can specifically bind to a polypeptide capable of forming an undesirable aggregate, conformation, or accumulation in a cell, and reducing or inhibiting this undesired polypeptide activity. Accordingly, the polypeptide binding molecules of the invention include small molecules, peptides, peptidomimetics, antibodies, antibody fragments, and intrabodies. The antibodies of the invention are typically administered as a polypeptide-based therapeutic but may also be administered in the form of an expressible nucleic acid. Alternatively, in another embodiment of the invention, the polypeptide binding molecule of the invention is an endogenous antibody produced by the host itself in response to the administration of a vaccine comprising either a polypeptide- or peptide-based antigen or a nucleic acid encoding such an antigen (i.e., a nucleic acid vaccine). In a most preferred embodiment, however, the polypeptide binding molecule of the invention is an intrabody.

Intrabodies

The intrabodies of the present invention are capable of binding a polypeptide that forms undesirable intracellular accumulations or aggregations. In one embodiment, the intrabodies bind to a polypeptide associated with a neurological disease, e.g., Alzheimer's disease, Parkinson's disease, Huntington's disease, prion disease, frontotemporal dementia, amyotrophic lateral sclerosis, spinal and bulbar muscular atrophy, dentatorubral-pallidoluysian atrophy, spinocerebellar ataxia type 1, spinocerebellar ataxia type 2, spinocerebellar ataxia type 3, spinocerebellar ataxia type 4, spinocerebellar ataxia type 5, spinocerebellar ataxia type 6, and spinocerebellar ataxia type 7.

The invention is intended to encompasses molecules such as intrabodies which are capable of binding to any polypeptide, wild type and/or abnormal mutant, having an association with a neurological disease involving an altered polypeptide, e.g., Amyloid Precursor Protein, Presenilin 1, Presenilin 2, α-2 Macroglobulin, an Apolipoprotein, α-Synuclein, huntingtin, a prion protein, Tau, Super oxide dismutase (SOD), Androgen receptor (AR), Atrophin 1, Ataxin 1, Ataxin 2, Ataxin 3, CACNL1A4, and SCA7. In addition, the invention is also intended to encompass intrabodies capable of binding to any polypeptide having an expanded polyglutamine region, e.g., AR, huntingtin, Atrophin 1, Ataxin 1, Ataxin 2, Ataxin 3, CACNL1A4, and SCA7 (see, e.g., Hurtley, S., Science 282:1071 (1998); Shoulson, I., Science 282:1072-1074 (1998); Hardy et al., Science 282:1075-1079 (1998); and Price et al., Science 282:1079-1083 (1998)).

In one embodiment, the invention provides an altered polypeptide representative of a polypeptide associated with disease and using the methods described in any one of U.S. Pat. Nos. 5,132,405; 5,091,513; 5,084,398; 5,525,491; and 5,851,829, or international patent application WO99/14353 and engineering intrabodies that can recognize an epitope specific to an altered polypeptide associated with a disease as compared to the corresponding normal polypeptide (see also, Zanetti et al, The Antibodies, Harwood Academic Pub. 4:1-141 (1997); Chen et al., Human Gene Therapy 5:595-601 (1994); Jones et al., Ad. Drug Delivery Reviews 31:153-170 (1998)). In a preferred embodiment, the intrabody is capable of binding to the huntingtin polypeptide. The binding could, for example, be specific for all huntingtin, normal and abnormal which accumulates and aggregates in neuronal cells to cause Huntington's disease.

Typically, such an engineered intrabody of the invention comprises a minimal sFv antibody comprising the variable region fragment (Fv) against an epitope on any of the polypeptides referred to herein. The Fv, sFv, or minimum antibody fragment which contains a complete antigen recognition and binding site, consists of a heterodimer of one heavy and one light chain variable domain in tight, noncovalent association. It is in this configuration that the three complementarity determining regions of each variable domain interact to define an antigen binding site on the surface of the V_{H}-V_{L} dimer. Collectively, the six complementarity determining regions (CDRs) confer antigen binding specificity to the intrabody.

This knowledge of the structure of immunoglobulin polypeptides has now been exploited to develop Fab, V_{H}, Fv, Fd, or single-chain Fv (sFv) antibody having a combining site/s that associates with any polypeptide that forms undesirable intracellular accumulations or aggregations. These antibodies are intended to function inside a cell and are therefore referred to as intrabodies.

Intrabodies with a Selected Affinity

The binding sites of the intrabodies embodying the invention are or may be biosynthetic in the sense that they are synthesized in a cellular host made to express a synthetic DNA, that is, a recombinant DNA obtained by the polymerase chain reaction and/or made from ligation of plural DNA segments, which may also be chemically synthesized oligonucleotides, or by ligation of fragments of DNA derived from the genome of a hybridoma, mature B cell clone, a cDNA library, or a phage library comprising nucleic acids derived from a mammal. In a preferred embodiment, the phage library is derived from a mammal that has either been immunized against a selected antigen (an immunized library) or a mammal that has not been expressly immunized against a given antigen (a naive library). In one embodiment, the mammal may be immunized against a particular antigen using a DNA vaccination using methods know in the art. Alternatively, the antigen-specific phage antibody may be selected from a naive phage display library. Thus, the intrabodies of the invention are designed specifically to have specificity and affinity for a preselected antigenic substance. The intrabodies of the invention are antibody-like in that their structure is patterned after regions of native antibodies known to be responsible for antigen recognition.

More specifically, the structure of these intrabodies in the region which imparts the binding properties to the polypeptide, is analogous to the Fv region of a natural antibody. Therefore, the intrabodies of the invention may be designed with the particular planned utility of the polypeptide in mind and comprise the CDRs or analogous CDRs or a portion thereof of the hypervariable region (and certain flanking amino acids) of an antibody having a known affinity and specificity, e.g., a murine, rat, or human monoclonal antibody to the selected polypeptide.

In another embodiment, the invention provides phage display methods for selecting appropriate intrabody binding sites ex vivo without the need for antigen processing and other complexities of the immune response. This method affords the ability to control the solvent conditions and method of antigen presentation (e.g., free in solution, in the presence of a co-solvent such as glycerol or sucrose, bound to magnetic beads, or adsorbed to plastic microtiter plates).

In one embodiment, the intrabodies of the invention uses the minimal binding site of human antibodies in the form of a single-chain Fv, wherein the H and L chain variable genes are fused at the gene level. The construction of such intrabodies is facilitated by selecting a binding site from a large, e.g., 7×10⁹ member, phage library of human sFv binding sites. These sFv genes have been typically ligated to the minor coat protein (gene 3 protein) of M13 bacteriophage, and reassembled into viable phage with the sFv-gene 3 fusions displayed at the tip of the filamentous phage. Thus, phenotype (i.e., binding site specificity and affinity) and genotype are linked in every phage particle. This method affords the advantage of conducting a genetic selection of a binding site in the absence of the immune system (see, e.g., Examples 4 and 5).

Moreover, after about several rounds of panning (phage selection, infection, and regrowth of infected cells), the method has the added advantage of allowing for the amplification of a selected phage which can be grown in a bacterial culture under conditions that produce the corresponding sFv in sufficient amounts for binding studies.

If larger preparations are desired, after the third round of selection, the phage can be grown from individual plaques by infection of an E. coli strain that yields free sFv that may be purified in vitro by IMAC chromatography directed to, e.g., any epitope or fused immunotag (e.g., the myc epitope (i.e., 9E10) or His₆ epitope), assessed for monomer or dimer forms present by FPLC on a Superose 6 column in PBS, and analyzed against the selected polypeptide, for example, a target polyglutamine biopolymer, which has been immobilized on streptavidin coated microchips in the BIAcore 2000 biosensor instrument. This biosensor instrument gives a measure of the association rate constant (Ka) and dissociation rate constant for the binding reaction (Kd), and the association equilibrium constant, Ka, calculated from the quotient of Ka/Kd. Accordingly, solvent conditions delineated in such physicochemical studies and used in phage selection can be designed to preserve relevant polypeptide conformation, e.g., during BIAcore measurements.

Bi-/Multi-Functional Intrabodies

In addition, the intrabodies of the invention may further comprise additional polypeptide regions defining a bioactive region, e.g., a targeting signal, enzyme sequence, toxin, or a site onto which a toxin or a remotely detectable substance can be attached, such as a therapeutic drug, binding polypeptide, enzyme or enzyme fragment, or an imaging agent.

In a preferred embodiment, the intrabody comprises another functional domain such as an enzyme, e.g., a protease, which can lead to the proteolysis of the polypeptide. In another embodiment, the intrabody comprises a targeting signal, e.g., ubiquitin, which can target the polypeptide to a proteasome for subsequent destruction. In yet another embodiment, the intrabody of the invention comprises a targeting signal that is capable of retargeting the intrabody bound polypeptide to another cellular locale. For, example, such a locale may be, e.g., cytoplasmic, nuclear, lysosomal, plasma membrane-associated, endoplasmic reticulum-associated, peroxisomal, or proteasomal. In addition, the intrabodies or binding molecules of the invention may encompass any art recognized targeting signal for altering the cellular location of a heterologous polypeptide.

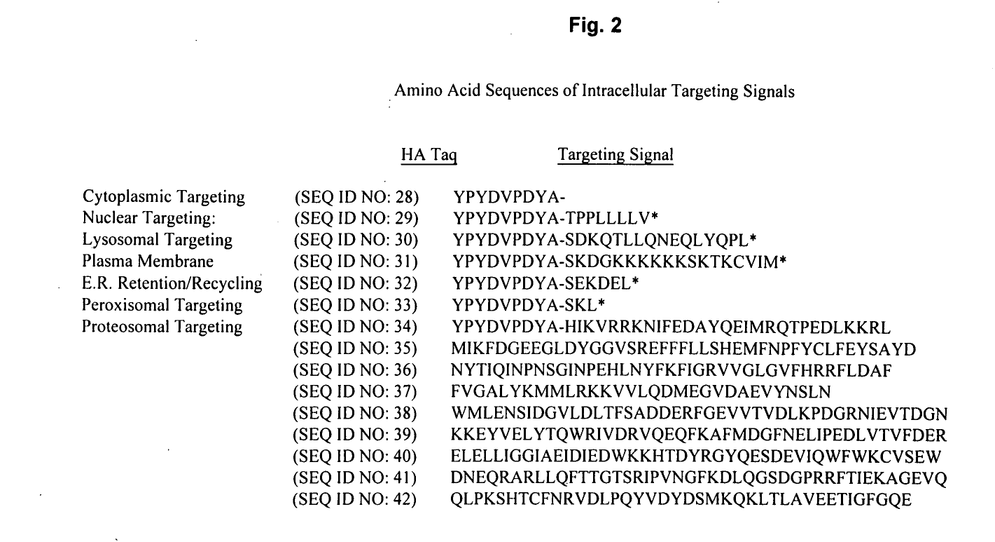

In one embodiment, the intrabody may be engineered to contain any art recognized targeting signal (e.g., as described in WO 99/14353 or shown in FIG. 2) suitable for targeting a polypeptide to a particular cellular location.

Multivalent Intrabodies

Of course, the intrabodies of the invention may comprise more than one binding site or copies of a single binding site, and a number of other functional regions. For example, multivalent intrabodies are included within the scope of the invention and such antibodies may have an affinity for one or more epitopes found within the selected polypeptide. Moreover, in a preferred embodiment, the multivalent intrabody may have one or more affinities for an epitope found within a normal peptide in addition to one or more affinities to an epitope found in an altered polypeptide. In another embodiment, the intrabody has affinity for one or more epitopes found within the altered polypeptide, for example, a polypeptide associated with disease. In another embodiment, the multivalent intrabody has the ability to selectively bind an altered polypeptide which may have a different half-life than that of the normal polypeptide, accumulate at a different rate or in a different cellular space (or be secreted), assume an altered conformation, aggregate (with itself or other polypeptides), form undesired interactions with other polypeptides, cause altered cell growth or cell death, and/or cause a disorder or disease.

Accordingly, in a preferred embodiment, the intrabody of the invention has one or more binding sites with affinity to any one or more of such polypeptides as, for example, Amyloid Precursor Protein, Presenilin 1, Presenilin 2, α-2 Macroglobulin, Apolipoprotein, α-Synuclein, huntingtin, Prion protein, Tau, SOD, AR, Atrophin 1, Ataxin 1, Ataxin 2, Ataxin 3, CACNL1A4, and SCA7.

In another preferred embodiment, the intrabodies of the invention are directed to any polypeptide involved in a disorder or disease that is neurological in nature such as, for example, Alzheimer's disease, Parkinson's disease, Huntington's disease, a prion disease, frontotemporal dementia (FTD), amyotrophic lateral sclerosis (ALS), spinal and bulbar muscular atrophy (SBMA or Kennedy disease), dentatorubral-pallidoluysian atrophy (DRPLA), spinocerebellar ataxia type 1 (SCA1), spinocerebellar ataxia type 2 (SCA2), spinocerebellar ataxia type 3 (SCA3 or Machado-Joseph disease (MJD)), spinocerebellar ataxia type 4 (SCA4), spinocerebellar ataxia type 5 (SCA5), spinocerebellar ataxia type 6 (SCA6), or spinocerebellar ataxia type 7 (SCA7).

Moreover, the intrabodies of the invention can be engineered to distinguish between a normal polypeptide having a short polyglutamine rich region and an expanded polypeptide rich region which may be associated with a disease state. To differentiate between polyglutamines that are only different in being short or long, intrabodies having a low affinity binding site or multiple low affinity binding sites may be used to preferentially bind the altered polypeptide (see, e.g., Huston, J. S., et al. (1992) Biophysical Journal, 62:87-91; Huston, J. S. et al., (1996), Adv. in Prot. Chem., 49:329-450). Thus, the invention encompasses divalent or multivalent forms of the sFv region of an intrabody that incorporate two or more binding sites in the smallest possible species (George A. J. and Huston, J. S., (1997), The Antibodies, 4:99-141; Huston J. S., et al. (1996) Quarterly Journal of Nuclear Medicine, 40:320-333). In addition, other domains may also be employed e.g., tetrameric streptavidin fusions, to facilitate the ability of the intrabodies to specifically target a selected polypeptide (Kipriyanov, S. M. et al., (1996) Protein Engineering 9:203-211).

In a preferred embodiment, the Ka of an intrabody, e.g., a univalent intrabody or binding molecule, is 10⁵ M⁻¹ and more preferably 10⁶ M⁻¹. In another embodiment, the Ka of a given valence of a multivalent intrabody or binding molecule is 10² M⁻¹, and more preferably, 10³ M⁻¹.

As is evidenced from the foregoing, the invention provides a large family of intrabodies comprising binding site/s patterned after the variable region or regions of natural immunoglobulins. It will be apparent that the nature of any polypeptide fragments linked to the intrabody, and used for reagents embodying the invention, are essentially unlimited, the essence of the invention being the provision, either alone or linked in various ways to other polypeptides, of being able to bind to a selected polypeptide, preferably, e.g., an altered polypeptide, that assumes an inappropriate cellular function.

Nucleic Acids Encoding Intrabodies

The intrabodies of the invention may be designed at the DNA level. Accordingly, nucleic acids encoding the intrabodies described herein are also within the scope of the invention.

The phage display library provides a collection of expressible nucleic acids whose expression and/or propagation is facilitated by phage-packaged nucleic acid sequences. In sFv-phage display libraries, the sFv is fused to a phage coat protein so that the sFv antibody combining site (the specific binding of which is the phenotype) is available for antigen binding on the phage surface, and its genotype (the DNA encoding the sFv) is contained within the phage antibody genetic material. This genotypic DNA is readily isolated, e.g. by restriction cleavage at SfiI and NotI sites, allowing amplification and sequencing, as well as subsequent manipulation of the sFv gene for use as an intrabody. The gene may be spliced into a pHEN1 plasmid for expression in E. coli, and the polypeptide expressed and secreted into the periplasm as a native folded polypeptide.

The ability to design the intrabody of the invention depends on the ability to determine the sequence of the amino acids in the variable region of monoclonal antibodies of interest, or the DNA encoding them. Hybridoma technology enables production of cell lines secreting antibody to essentially any desired substance that produces an immune response. RNA encoding the light and heavy chains of the immunoglobulin can then be obtained from the cytoplasm of the hybridoma, and the 5′ end portion of the mRNA can be used to prepare the cDNA for subsequent sequencing, or the amino acid sequence of the hypervariable and flanking framework regions (FR) can be determined by amino acid sequencing of the H and L chains and their V region fragments. Such sequence analysis is now conducted routinely. This knowledge permits one to design synthetic genes encoding FR and CDR sequences which likely will bind the antigen. These synthetic genes are then prepared using known techniques, or using the technique disclosed below, and then inserted into a suitable host, expressed, and purified. Depending on the host cell, renaturation techniques may be required to attain proper conformation. The various polypeptides are then tested for binding ability, and one having appropriate affinity is selected for incorporation into a reagent of the type described above. If necessary, point substitutions seeking to optimize binding may be made in the DNA using conventional cassette mutagenesis or other protein engineering methodology.

Of course, the processes for manipulating, amplifying, and recombining DNA which encode amino acid sequences of interest are generally well known in the art, and therefore, not described in detail herein. Methods of identifying and isolating genes encoding intrabody regions of interest are well understood, and described herein and in, e.g., U.S. Pat. Nos. 5,132,405; 5,091,513; 5,084,398; 5,525,491; 5,851,829, and international patent application WO99/14353 and these references are hereby incorporated by reference herein.

Intrabody Antigens—Target Polypeptides

An isolated target polypeptide, preferably a polypeptide known to form undesired intracellular accumulations or aggregations, more preferably a polypeptide expressed at comparatively higher concentrations in the brain or in cells of neuronal origin than elsewhere, or which causes associated non-neuronal disorders, or a portion or fragment thereof, can be used as a target for phage library selection procedures, or as an immunogen to generate intrabodies that bind the target polypeptide using standard techniques for polyclonal and monoclonal antibody preparation. A full-length target polypeptide can be used or, alternatively, the invention provides antigenic peptide fragments of the target polypeptide, e.g., the huntingtin N-terminal sequence (e.g., residues 1-17), for use as immunogens or targets in phage display selection. Preferably, the antigenic peptide encompasses an epitope of the target polypeptide that is formed by at least 5 amino acid residues, more preferably by at least 15 amino acid residues, such that an intrabody raised against the peptide forms a specific immune complex with the target polypeptide under native conditions.

Alternatively, a polypeptide representing a polyglutamine rich region that may be common to any number of altered polypeptides may be used as the target antigen (e.g., huntingtin and Atrophin). Such a polypeptide allows for the raising or selecting of an intrabody that can bind any number of polypeptides having a polyglutamine rich region. Accordingly, such an intrabody could inhibit the accumulation, formation of aggregates, or retarget the cellular localization, of several forms of an altered polypeptide if each form had a minimal polyglutamine rich region. However, specificity for the unique class of polypeptides of interest, e.g. huntingtin, preferably requires a bispecific or multi-specific sFv antibody or diabody or related forms of the Fv, wherein additional specificity is conferred for non-polyglutamine, polypeptide-specific epitopes The diabody is a bivalent or bispecific, or sometimes even trivalent and multipsecific, form of the single-chain Fv that forms when the linker connects the V domains is abnormally short, usually 10 residues or fewer, and preferably 5 residues or fewer, resulting in the formation of Fv binding sites between the variable domain fusion proteins.

A chosen polypeptide immunogen typically is used to select intrabodies from a phage library or to elicit antibodies by immunizing a suitable subject, (e.g., rabbit, goat, mouse, or other mammal, including, e.g., a human patient) with the target polypeptide immunogen. An appropriate immunogenic preparation can contain, for example, recombinantly expressed target polypeptide or a chemically synthesized polypeptide.

The polypeptide may be further fused to a moiety to facilitate detection or purification and such moieties include immunoreactive tags, GFP, biotin, etc. The preparation can further include an adjuvant, such as Freund's complete or incomplete adjuvant, or similar immunostimulatory agent.

Alternatively, the immunogen may be administered in the form of an expressible nucleic acid, e.g., as a DNA vaccine, such that the nucleic acid is taken up and expressed by cells of the recipient animal, such that a polypeptide in a sufficient amount to provoke a host cell immune response, preferably a humoral response, is achieved. Immunization of a suitable subject with an immunogenic target polypeptide preparation induces an anti-target polypeptide antibody response.

Accordingly, the intrabody may be derived from a monoclonal antibody raised using standard techniques. More preferably, the intrabody is derived from an animal that has been immunized to the target polypeptide using DNA vaccination. Even more preferably, the intrabody is derived from a phage library that is derived from an animal that has been immunized with the target polypeptide or an expressible DNA encoding the target polypeptide. In certain cases, the in situ production of antibodies to the target polypeptide may yield intact IgG or other classes of antibody that enter diseased cells to act as intrabodies, thereby counteracting the pathological aggregation of the target polypeptide, whether in neuronal cells or other cells that suffer from collateral disease derived from the same target polypeptide. In these cases, the therapeutic antibody may be administered as a polypeptide immunotherapy or as a form of adoptive immunotherapy in which cells modified ex vivo are readministered to the patient, where they secrete the intrabody polypeptide (see, e.g. Cavazzana-Calvo, M, et al., Science 288: 669-672).

In addition, an intrabody can be identified and isolated by screening a recombinant combinatorial immunoglobulin library (e.g., an intrabody phage display library) with the target polypeptide to thereby isolate immunoglobulin library members that bind the target polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating, screening, and selecting intrabodies of a particular affinity can be found in, for example, U.S. Pat. Nos. 5,132,405; 5,091,513; 5,084,398; 5,525,491; 5,851,829, international patent application WO99/14353, and Sheets et al., P.N.A.S. 95:6157-6162 (1998).

Thus, the present invention shows (as the Examples infra will further evidence) at least the following intrabody concepts and they are: 1) that intrabodies can be delivered as a gene therapy; 2) that intrabodies can be delivered as a protein immunotherapy that enters the target cell (like an immunotoxin) and functions through binding the target antigen within the cell; 3) that intrabodies, e.g., in the form of a host humoral response, can be elicited by appropriate immunization, such that the intrabody protein for immunotherapy is made by the immune system instead of being administered intravenously; and 4) abnormal polypeptide accumulations can be present at very low levels and still significantly disrupt cellular function via protein-protein interactions and thus, even modest intracellular levels of intrabodies are therapeutic.

III. Expression Vectors and Host Cells

Another aspect of the invention pertains to vectors, preferably expression vectors, containing a nucleic acid encoding an intrabody (or a portion thereof). One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, lentiviruses, adenoviruses and adeno-associated viruses), which serve equivalent functions.

The recombinant expression vectors of the invention comprise nucleic acids encoding an intrabody of the invention in a form suitable for expression in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, inducible promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce intrabodies that may then be administered as a polypeptide formulation. Alternatively, the nucleic acid encoding an intrabody is preferably introduced into a vector that can be expressed in a mammal, more preferably, in a human, and more preferably, in a cell of neuronal origin in a mammal.

A number of promoters and expression vectors are known in the art as affording high expression in mammalian cells. For example, a nucleic acid of the invention can be expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells (see Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., (1989); Ausubel et al., (1998) Current Protocols in Molecular Biology, John Wiley & Sons, Inc.).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calarne and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, intrabody protein can be expressed in bacterial cells such as E. coli, insect cells, yeast or mammalian cells (such as, e.g., Chinese hamster ovary cells (CHO), mammalian kidney cells (COS), hamster kidney cells (BHK-21), human epithelial cells (293T)). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. In a preferred embodiment, nucleic acids encoding intrabodies may be transfection using protamine-based non-viral vectors (Richardson J. H., (1995) Trends In Biotechnology, 13:306-310). Suitable methods for transforming or transfecting host cells can be found in Sambrook, et al., above, and, e.g., other laboratory manuals and references referred to herein.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an intrabody or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) the intrabody. Accordingly, the invention further provides methods for producing intrabody polypeptides using the host cells of the invention. In one embodiment, the method comprises culturing the host cell of invention (into which a recombinant expression vector encoding the intrabody has been introduced) in a suitable medium such that the intrabody is produced. In another embodiment, the method further comprises isolating the intrabody from the medium or the host cell.

The host cells of the invention can also be used to produce nonhuman transgenic animals. For example, in one embodiment, a host cell of the invention is a fertilized oocyte or an embryonic stem cell into which intrabody-coding sequences have been introduced. Such host cells can then be used to create non-human transgenic animals in which exogenous intrabody sequences have been introduced into their genome or homologous recombinant animals in which endogenous Fv sequences have been altered. Such animals are useful for studying the function and/or activity of an intrabody and for identifying and/or evaluating modulators of intrabody/target polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous target polypeptide gene has been altered by homologous recombination between the endogenous gene and an exogenous DNA molecule introduced into a cell of the animal, e.g., an embryonic cell of the animal, prior to development of the animal. This animal may be further modified to also express an intrabody or can be bred against an animal that expresses an intrabody. More preferably, the transgenic animal is engineered such that one or more of the polypeptides of interest, i.e., either the target polypeptide, or more preferably, the intrabody, is under conditional control as, e.g., as described in Example 6. In addition, any art recognized techniques may be used to produce a transgenic animal and/or homologous recombinant animal of the invention.

IV. Screening Assays

The invention provides a method (also referred to herein as a “screening assay”) for identifying modulators, i.e., candidate binding molecules (e.g., peptides, peptidomimetics, small molecules, or other drugs) which bind to the target polypeptide, e.g., a huntingtin polypeptide. The method is conducted in situ, within cells that exhibit abnormalities that present a model of the given disease. Thus the ability to counteract pathological aggregation of the target polypeptide in situ, provides a prerequisite for further therapeutic development.

In particular, one method of the invention involves the use of an intrabody in a cell of a non-human subject for the screening of compounds that alter the undesired accumulation, complexing, or aggregation of a selected polypeptide. In a preferred embodiment the selected polypeptide is, e.g., Amyloid Precursor Protein, Presenilin 1, Presenilin 2, α-2 Macroglobulin, Apolipoprotein, α-Synuclein, huntingtin, a prion protein, Tau, Superoxide dismutase (SOD), Androgen receptor (AR), Atrophin 1, Ataxin 1, Ataxin 2, Ataxin 3, CACNL1A4, or SCA7. In another preferred embodiment, the polypeptide is associated with Alzheimer's disease. In yet another preferred embodiment, the polypeptide is huntingtin, abnormal forms of huntingtin being involved in Huntington's disease. Accordingly, an intrabody specific for the polypeptide involved in a particular disease is brought into contact with the polypeptide in vitro, in a cell-based assay, or in the context of a whole animal, and one or more compounds is administered on or about the same time and a change in aggregation due to the intrabody/polypeptide interaction is monitored.

In vitro, such a change may be measured as a change in binding or some other biochemical parameter. In the cell-based assay, the alteration in the intrabody/polypeptide interaction may be measured as a change in the accumulation, complexing, or aggregation of the target polypeptide or a change in the cell biology of the host cell (e.g., a change in levels of cell death). In the context of a whole organism, such an alteration of the in vivo interaction of the intrabody with target polypeptide may be measured as an alteration in disease symptomatology.

One or all of the assays may be used to confirm the potential therapeutic efficacy of a compound that alters accumulation of aggregates by virtue of the intrabody/polypeptide interaction. Such a compound may then be selected for further analysis. Ideally, in a preferred embodiment, the compound is identified as specifically binding to the target polypeptide. In another preferred embodiment, the compound can recognize both the normal and mutant polypeptide, and by complexing them at an appropriate level, promote both normal cell function and elimination of pathological processes. In still another preferred embodiment, the compound can recognize a mutant polypeptide with a higher specificity than a wild type polypeptide or can be altered to function as thus. In yet another preferred embodiment, the compound may be selected for certain pharmacological properties or advantages such as, e.g., toxicity, ability to cross the blood-brain-barrier, half-life, ability to be linked to another functional moiety, or ability to be further modified.

The binding molecule of interest may also be assayed using tissue slice cultures, e.g., organotypic slice cultures from an animal, e.g., a transgenic animal model for a neurological disease (e.g., AD, HD, a SCA disease, etc.). In a preferred embodiment, mice having HD or SCA1 are employed. In addition, any of the foregoing experimental animals can be tested in parallel with wild type controls for their sensitivity to compounds such as excitotoxins or those known to cause oxidative damage. A number of chemicals described in the art that are suitable for inducing neurodegeneration, e.g., HD-like degeneration of the striatum, or other neurotoxic effects can be tested in vivo or by using organotypic slice cultures derived from such animals (see Table 1 and, e.g., Bowling et al., Life Sci. 56:1151-1171 (1995); Jansel et al., Brain Res. 532:351-354 (1990); Rothstein et al. PNAS 90:6591-6595 (1993); and Schwarcz et al., Life Sci. 35:19-32 (1963)). By using doses that are not overtly toxic to wild-type cells or tissue following short-term exposure, differential vulnerability of diseased cells can be ascertained due to counteracting the early, subclinical pathology that would normally be elicited.

In a preferred embodiment, the compounds described in Table 1 may be used to elicit differential effects in either mutant neurons, wild type neurons, or neurons having an intrabody or binding molecule.

TABLE 1

| | Inhibitors of | | |
|---|---|---|---|
| | mitochondrial | Inhibitors of | |
| Metal chelators | function | calmodulin | Excitotoxins |
| a. DDC | a. Myxothiazol: | a. Calmidazonium: | a. Quinolinic acid: |
| (diethyidithiocarbamide): | 10⁻⁷-10⁻⁹ M | 10⁻⁵-10⁻⁷ M | 1-100 μM |
| 10⁻³-10⁻⁶ M | | | |
| b. EGTA: same | b. 3-nitroproprionic | b. Trifluoperazine: | b. Kainic acid: |
| | acid: 10⁻³-10−6 M | 10⁻⁴-10⁻⁶ M | 1-100 μM |
| | c. Malonate: | | |
| | 1-100 mM | | |

In one embodiment, cultures can be prepared using the methods as described herein, and allowed to mature for two weeks. Although recognizable pathology due to, e.g., the presence of either the HD or the SCA1 transgenes should be minimal at this time, the neurons in the slices are still likely to be at increased risk. Compounds shown above can then be added and the results monitored morphologically.

Accordingly, this assay system is ideal for screening small molecule therapies. In one embodiment, a comparison of amino acid sequence differences among the sFv binding sites (e.g., complementarity determining regions, CDRs) specific for expanded repeats of the polyglutamine regions is made to find a structural basis for specific binding. In particular, the H3 CDR loop of the sFv specific for a huntingtin polypeptide is responsible for much of the intrabody/polypeptide binding interaction, and therefore H3 peptide mimetics may be screened for using this system and used in place of intact intrabodies. This information can then be used to identify other binding molecules that interact with aberrant polyglutamine regions such as conformational epitopes and these may be screened for using, e.g., peptide-phage display libraries (New England Biolabs) to find appropriate analogues.

In a preferred embodiment, the above assay is used to identify a candidate binding molecule having an inhibitory effect on polypeptide accumulation or aggregation. In another embodiment, the candidate compound may mimic the bioactivity of an intrabody, e.g., prevent aggregation and retarget the polypeptide.

The test compounds of the present invention can be obtained using any of the numerous approaches involving combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA. 90:6909; Erb et al. (1994) Proc. Natl. Acad Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad Sci. 87:6378-6382); (Felici (1991) J. Mol. Biol. 222:301-310); and (Ladnersupra.).

In one embodiment, an assay is a cell-based assay in which a cell which expresses a target polypeptide, e.g., huntingtin and an intrabody is contacted with a test compound and the ability of the test compound to alter the polypeptide/intrabody interaction is determined. The cell, for example, can be of mammalian origin or a yeast cell. Determining the ability of the test compound to bind to polypeptide can be accomplished, for example, by coupling the test compound with a radioisotope or enzymatic label such that binding of the test compound to the polypeptide can be determined by detecting the labeled compound in a complex. For example, test compounds can be labeled with ¹²⁵I, ³⁵S, ¹⁴C, ³H or ³²P, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with a target polypeptide without the labeling of any of the interactants. For example, a microphysiometer can be used to detect the interaction of a test compound with a target polypeptide without the labeling of either the test compound or the target polypeptide (McConnell, H. M. et al. (1992) Science 257:1906-1912).

In yet another embodiment, an assay of the present invention is a cell-free assay in which the target polypeptide and intrabody are contacted with a test compound and the ability of the test compound to alter the polypeptide/intrabody interaction is determined. Binding of the test compound to the target polypeptide can be determined either directly or indirectly. Determining the ability of the candidate compound to bind to the target polypeptide can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

In many drug screening programs which test libraries of compounds and natural extracts, high throughput assays are desirable in order to maximize the number of compounds surveyed in a given period of time. Assays which are performed in cell-free systems, such as may be performed using purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with upstream or downstream elements. Accordingly, in an exemplary screening assay of the present invention, the compound of interest is contacted with the target polypeptide and detection and quantification of accumulation or aggregation of the target polypeptide is determined by assessing a compound's efficacy at inhibiting the formation of undesired complexes or aggregates. The efficacy of the compound can be assessed by generating dose response curves from data obtained using various concentrations of the test compound. Moreover, a control assay can also be performed to provide a baseline for comparison.

In more than one embodiment of the above assay methods of the present invention, it may be desirable to immobilize the target polypeptide to facilitate separation of complexed from uncomplexed forms or accommodate automation of the assay. Binding of a test compound to a target polypeptide can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferas/target polypeptide fusion proteins can be adsorbed onto glutathione Sepharose beads (Sigma Chemical, St. Louis, Mo.) or glutathione derivatized micrometer plates, which are then combined with the test compound or the test compound and the intrabody and incubated under conditions conducive to complex formation (e.g., at physiological conditions of ionic strength and pH). Following incubation, the beads or micrometer plate wells are washed to remove any unbound components, the matrix immobilized in the case of beads, and the complex is measured either directly or indirectly, for example, as described above. Alternatively, the complexes can be dissociated from the matrix, and the level of target polypeptide binding or activity can be determined using standard techniques.

The testing of the compounds that are selected by such screening methods may be assessed for their relevance to a given neurological disease, such as Huntington's, using a hierarchy of models, starting with a transfected cell model, going on to brain slices from appropriate normal or mutant mice, and then with virally transduced mouse or transgenic mouse models of disease. By these mean, the test compounds may be narrowed down to those few which may merit clinical testing.

Other techniques for immobilizing proteins on matrices can also be used in the screening assays of the invention. For example, either the target polypeptide can be immobilized utilizing conjugation of biotin and streptavidin. Biotinylated target polypeptides or intrabodies can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In yet another aspect of the invention, the target polypeptide or the intrabody binding region can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins or compounds, which bind to or interact with the target polypeptide and/or the intrabody.

This invention further pertains to novel agents identified by the above-described screening assays and to processes for producing such agents by use of these assays. Accordingly, in one embodiment, the present invention includes a compound or agent obtainable by a method comprising the steps of any one of the aforementioned screening assays (e.g., cell-based assays or cell-free assays). For example, in one embodiment, the invention includes a compound or agent obtainable by any of the methods described herein.

Accordingly, it is within the scope of this invention to further use an agent, e.g., a compound or intrabody identified as described herein in an appropriate animal model. For example, an agent identified as described herein can be used in an animal model to determine the efficacy, toxicity, or side effects of treatment with such an agent. Alternatively, an agent identified as described herein can be used in an animal model to determine the mechanism of action of such an agent. In addition, such an agent if deemed appropriate, may be administered to a human subject, preferably a subject at risk for a neurological disorder.

The present invention also pertains to uses of novel agents identified by the above-described screening assays for diagnoses, prognoses, and treatments of any of the neurological disorders described herein. Accordingly, it is within the scope of the present invention to use such agents in the design, formulation, synthesis, manufacture, and/or production of a drug or pharmaceutical composition for use in diagnosis, prognosis, or treatment of any of the neurological disorders described herein.

V. Methods of Use

Prophylactic Methods

In one aspect, the invention provides a method for preventing in a subject, a disease or condition associated with an aberrant or undesired intracellular accumulation or aggregation of a neuronal polypeptide, by administering to the subject an agent which inhibits the accumulation of the polypeptide or the formation of such complexes or aggregates. In a preferred embodiment, the disease or condition is a neurological condition (e.g., see Table 2, taken from Hardy et al., Science 282:1075-1079 (1998)). Subjects at risk for a neurological disease which is caused or contributed to by such aberrant or undesired polypeptide activity can be identified by, for example, art recognized diagnostic or prognostic techniques. Accordingly, upon presentation of a positive diagnosis, administration of any of the appropriate compounds or intrabodies described herein may be administered as a prophylactic agent prior to the manifestation of symptoms characteristic of the aberrancy, such that the neurological disease or disorder is prevented, lessened in its severity, or delayed in its progression or onset.

TABLE 2
Autosomal dominant primary neurodegenerative diseases.

| | | | | | Transgenic | |
|---|---|---|---|---|---|---|
| Disease | Linkage | Gene | Mutations | Pathology | (Comment) | Ref. |
| Prion | Ch20 | Prion | Mainly missense | PrP plaques, | +(no T or LB) | (1) |
| | | | | sometimes T or | | |
| | | | | LB; classically | | |
| | | | | associated with | | |
| | | | | spongiform | | |
| | | | | changes | | |
| AD | Ch21 | APP | Missense around Aβ, | Amyloid | +(no T or LB) | (2) |
| | | | increase Aβ42 | plaques and T, | | |
| | | | | may see LB | | |
| | Ch14 | PS1 | Mainly Missense, | Amyloid | +(no plaques T | (3) |
| | | | increase Aβ42 | plaques and T | or LB) | |
| | Ch1 | PS2 | Missense, increase | Amyloid | +(no plaques T | (4) |
| | | | Aβ42 | plaques and T | or LB) | |
| PD | Ch4q | α-synuclein | Missense | LB | Not reported | (5) |
| | Ch2 | Not identified | Not known | LB (and T?) | Not reported | (6) |
| | Ch4p | Not identified | Not known | LB | Not reported | (7) |
| FTD | Ch17 | Tau | Missense and splice | T, sometimes | Not reported | (8) |
| | | | | with “unusual | | |
| | | | | periodicity” | | |
| | Ch3 | Not identified | Not known | Not reported | Not reported | (9) |
| ALS | Ch21 | SOD | Mainly missense | Lewy-like | +(motor neuron | (10) |
| | | | | bodies | disease, | |
| | | | | | inclusions, cell | |
| | | | | | loss) | |
| SBMA* | X | AR | Polyglutamine | Nuclear | +(no | (11) |
| | | | | inclusions | phenotype) | |
| HD | Ch4 | huntingtin | Polyglutamine | Nuclear | +(inclusions, | (12) |
| | | | | inclusions | movement | |
| | | | | | disorder, cell | |
| | | | | | loss) | |
| DRPLA | Ch12 | Atrophin 1 | Polyglutamine | Nuclear | Not reported | (13) |
| | | | | inclusions | | |
| SCA1 | Ch6 | Ataxin 1 | Polyglutamine | Nuclear | +(ataxic. | (14) |
| | | | | inclusions | inclusions, cell | |
| | | | | | loss) | |
| SCA2 | Ch12 | Ataxin 2 | Polyglutamine | Not reported | Not reported | (15) |
| SCA3/MJD | Ch14 | Ataxin 3 | Polyglutamine | Nuclear | +(ataxic. | (16) |
| | | | | inclusions | cerebellar | |
| | | | | | atrophy) | |
| SCA4 | Ch16 | Not identified | Not known | Not reported | Not reported | (17) |
| SCA5 | Ch11 | Not identified | Not known | Not reported | Not reported | (18) |
| SCA6 | Ch19 | CACNL1A4 | Polyglutamine | Not reported | Not reported | (19) |
| SCA7 | Ch3 | SCA7 | Polyglutamine | Nuclear | Not reported | (20) |
| | | | | inclusions | | |

Ch, chromosome;
PrP, prion protein;
T, tangles;
LB, Lewy bodies;
+, is present or exists;
AD, Alzheimer's Disease;
PD, Parkinson's Disease;
HD, Huntington's Disease;
SOD, Superoxide dismutase.
*SBMA is technically not autosomal dominant but it is probably dominant in its cellular mode of action.

Therapeutic Methods

Another aspect of the invention pertains to methods of treating an individual afflicted with a disease or disorder characterized by aberrant expression or activity of a target polypeptide, preferably a polypeptide expressed in a neuronal cell.

Accordingly, the invention provides methods for treating diseases or conditions associated with a cell of the central or peripheral nervous system. For example, the invention provides methods for treating lesions of the nervous system associated with aberrant polypeptide accumulation or aggregation that may, for example, lead to abnormal cell proliferation, differentiation, or cell death of any of the following cells: neurons (including, e.g., motor neurons, sensory neurons), Schwann cells, Purkinje cells, astrocytes, microglial cells, ependymal cells, oligodendrocytes or any other types of neural cells. Disorders of the nervous system include, but are not limited to, Alzheimer's disease, Parkinson's disease, Huntington's disease, a prion disease, frontotemporal dementia (FTD), amyotrophic lateral sclerosis (ALS), spinal and bulbar muscular atrophy (SBMA or Kennedy disease), dentatorubral-pallidoluysian atrophy (DRPLA), spinocerebellar ataxia type 1 (SCA1), spinocerebellar ataxia type 2 (SCA2), spinocerebellar ataxia type 3 (SCA3 or Machado-Joseph disease (MJD)), spinocerebellar ataxia type 4 (SCA4), spinocerebellar ataxia type 5 (SCA5), spinocerebellar ataxia type 6 (SCA6), and spinocerebellar ataxia type 7 (SCA7).

Inhibiting the Formation of Undesired Intracellular Polypeptide Complexes/Accumulations

In one embodiment, the invention provides methods for inhibiting the formation of undesired intracellular polypeptide aggregates or accumulations. In particular, the intrabodies of the invention are designed to inhibit the formation of any undesired polypeptide from accumulating or forming a complex or aggregate by specifically binding to the polypeptide. In some circumstances, the mere specific binding of the intrabody to the polypeptide may be sufficient to inhibit the formation of complexes or aggregates comprising the polypeptide. Such an intrabody may have one or more valences, i.e., binding sites, with an affinity for the target polypeptide, and may, e.g., inhibit accumulation or aggregation of the polypeptide by steric hindrance, or by inducing an altered conformation that renders the protein less stable, and/or by rendering the complexed polypeptide less able to form interactions that lead to an accumulation or aggregation of the polypeptide, e.g. by increasing its solubility or increasing the critical concentration at which aggregates form.

Accordingly, the invention provides methods for administering to a subject any of the intrabodies described herein, either alone, or in combination with another intrabody, or other suitable therapeutic for targeting a polypeptide known to form undesired intracellular polypeptide accumulations or aggregates. In one embodiment, the subject may be an experimental animal. In a preferred embodiment, the methods of the invention are suitable for treating a human patient. In another preferred embodiment, the administration of an intrabody or binding molecule is for the treatment or cure of Alzheimer's Disease. In still another preferred embodiment, the administration of an intrabody or binding molecule is for the treatment of Huntington's disease. In one embodiment of the invention, the method of administering an intrabody is as a polypeptide. In a preferred embodiment of the invention, the intrabody is administered as an expressible nucleic acid in the form of gene therapy as described in the subsection below.

Gene Therapy

The intrabodies or peptide-based binding molecules of the invention are particularly useful in the treatment of any of the neurological diseases described herein when administered as gene therapy. The general approach involves the introduction of a nucleic acid encoding an intrabody or peptide-based binding molecule into cells such that one or more gene products encoded by the introduced genetic material are produced in the cells to inhibit undesired polypeptide accumulation or aggregation.

The nucleic acid molecules of the invention encoding intrabodies or peptide-based binding molecules that can be encoded with a nucleic acid and inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. The pharmaceutical compositions can be included in a container. Gene therapy vectors typically utilize constitutive regulatory elements which are responsive to endogenous transcriptions factors. In a preferred embodiments, the gene therapy vectors encoding the intrabody or peptide binding molecule is an expression vector derived from a virus that is an adenovirus, adeno-associated virus, retrovirus, or herpes simplex virus (see, e.g., During et al., Mol Med Today. 4:485-93 (1998); During et al., Nat Med. 4:1131-5 (1998); During et al., Gene Ther. 5:820-7 (1998); Kaplitt et al. Pediatr Neurosurg. 28:3-14 (1998); Freese et al., Epilepsia 38:759-66 (1997); O'Connor et al., Exp Neurol. 148:167-78 (1997); During et al., Exp Neurol. 144:74-81 (1997); Freese et al. Mov Disord 11:469-88 (1996); During, Lancet 348:618 (1996); Freese et al., J Clin Endocrinol Metab. 81:2401-4 (1996); During et al., Clin Neurosci. 3:292-300 (1995-6); During et al., Science 266:1399-403 (1994); and Kaplitt et al., Nat Genet. 8:148-54 (1994)).

Non-viral gene delivery vehicles are also a means to effect cell-specific delivery of the therapeutic plasmids for the present invention. These are traditionally antibodies or single-chain Fv antibodies that are coupled or fused to DNA complexing agents (see Uherek et al., J. Biol. Chem. 273:8835-8841 (1998); Foster et al., Human Gene Therapy, 8:719-727 (1997); Chen et al., Gene Therapy 2:116-123 (1995). This class of gene delivery vehicles also includes antibodies or their fragments coupled to liposomes (Huang et al., U.S. Pat. Nos. 4,925,661/4,957,735/6,008,202).

For reviews on gene therapy approaches see Anderson, W. F. (1992) Science 256:808-813; Miller, A. D. (1992) Nature 357:455-460; Friedmann, T. (1989) Science 244:1275-1281; and Cournoyer, D., et al. (1990) Curr. Opin. Biotech. 1:196-208.

For further descriptions of cell types, genes, and methods for gene therapy see e.g., Wilson, J. M et al. (1988) Proc. Natl. Acad Sci. USA 85:3014-3018; Armentano, D. et al. (1990) Proc. Natl. Acad. Sci. USA 87:6141-6145; Wolff, J. A. et al. (1990) Science 247:1465-1468; Chowdhury, J. R. et al. (1991) Science 254:1802-1805; Ferry, N. et al. (1991) Proc. Natl. Acad Sci. USA 88:8377-8381; Wilson, J. M. et al. (1992) J. Biol. Chem. 267:963-967; Quantin, B. et al. (1992) Proc. Natl. Acad. Sci. USA 89:2581-2584; Dai, Y. et al. (1992) Proc. Natl. Acad Sci. USA 89:10892-10895; van Beusechem, V. W. et al. (1992) Proc. Natl. Acad Sci. USA 89:7640-7644; Rosenfeld, M. A. et al. (1992) Cell 68:143-155; Kay, M. A. et al. (1992) Human Gene Therapy 3:641-647; Cristiano, R. J. et al. (1993) Proc. Natl. Acad. Sci. USA 90:2122-2126; Hwu, P. et al. (1993) J. Immunol. 150:4104-4115; and Herz, J. and Gerard, R. D. (1993) Proc. Natl. Acad. Sci. USA 90:2812-2816; Bachoud-Levi et al., (1998) Progress in Brain Research, 117:511-524; Lowenstein et al. (1998) Progress in Brain Research 117:485-501; Weyerbrock et al. (1999) Current Opinion in Oncology 11:168-173; Karpati et al. (1996) Trends Neurosci. 19:49-54; Skaper et al. (1998) Mol. and Cell. Neurosci. 12:179-193; Suhr et al. (1999) Arch. Neurol. 56:287-292; Rabinowitz et al. (1998) Current Opinion in Biotechnology 9:470-475; Tyler et al. (1999) P.N.A.S. 96:7053-7058.

VI. Pharmaceutical Compositions

The intrabodies of the invention can be incorporated into pharmaceutical compositions suitable for administration. Such compositions typically comprise the intrabody and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with the pharmaceutical administration of an intrabody or binding molecule in the form of, e.g., a polypeptide, nucleic acid, peptide-based binding molecule, or small molecule. The use of such media and agents for pharmaceutically active substances is well known in the art. Except insofar as any conventional media or agent is incompatible with the intrabody, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, intrathecal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the intrabody in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the intrabody can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The intrabodies can also be prepared in the form of suppositories (e.g., with conventional suppository bases such as cocoa butter and other glycerides) or retention enemas for rectal delivery.

In one embodiment, the intrabodies are prepared with carriers that will protect the intrabody against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of intrabody calculated to produce the desired therapeutic effect in association with the required pharmaceutical carrier. The specification for the dosage unit forms of the invention are dictated by and directly dependent on the unique characteristics of the intra body and the particular therapeutic effect to be achieved, and the limitations inherent in the art of compounding such an intrabody for the treatment of subjects.

Toxicity and therapeutic efficacy of such intrabodies can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Intrabodies which exhibit large therapeutic indices are preferred. While intrabodies that exhibit toxic side effects may be used, care should be taken to design a delivery system that targets such intrabodies to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any intrabody used in the method of the invention, the therapeutically effective dose can be estimated initially from cell culture assays. A dose may be formulated in animal models to achieve a circulating plasma concentration range that includes the IC50 (i.e., the concentration of the test intrabody which achieves a half-maximal inhibition of symptoms) as determined in cell culture. Such information can be used to more accurately determine useful doses in humans. Levels in plasma may be measured, for example, by high performance liquid chromatography.

The genes encoding the intrabodies of the invention can be inserted into vectors and used as gene therapy vectors. Gene therapy vectors can be delivered to a subject as described in the foregoing subsection.

The pharmaceutical compositions can be included in a container, pack, or dispenser together with instructions for administration.

This invention is further illustrated by the following examples which should not be construed as limiting.

### EXAMPLES

Throughout the examples the following materials and methods were used unless otherwise stated.

Materials and Methods

In general, the practice of the present invention employs, unless otherwise indicated, conventional techniques of chemistry, molecular biology, recombinant DNA technology, PCR technology, immunology (especially, e.g., antibody technology), and any necessary cell culture or animal husbandry techniques, which are within the skill of the art and are explained in the literature. See, e.g., Sambrook, Fritsch and Maniatis, Molecular Cloning: Cold Spring Harbor Laboratory Press (1989); DNA Cloning, Vols. 1 and 2, (D. N. Glover, Ed. 1985); PCR Handbook Current Protocols in Nucleic Acid Chemistry, Beaucage, Ed. John Wiley & Sons (1999); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); Current Protocols in Molecular Biology, eds. Ausubel et al., John Wiley & Sons (1992); Large-Scale Mammalian Cell Culture Technology, Lubiniecki, A., Ed., Marcel Dekker, Pub., (1990); and Manipulating the Mouse Embryo, Hogan et al., C.S.H.L. Press, Pub (1994).

Construction of GST and GFP-Fusion Proteins

Standard techniques were used for all plasmid constructions (see, e.g., Ausubel et al., and Sambrook et al., supra).

DRPLA constructs are from Onodera et al., or derived as described by Onodera et al. (FEBS letters (1996) 399: 135-139).

HD constructs, e.g., HD-Q25 and HD-Q104 cDNAs were amplified from pcDNA-HD17 and pcDNA-HD14, respectively (gifts of A. Kazantsev et al., Proc. Natl. Acad. Sci. 96:11404-11409 (1999), by PCR (25 cycles of 95° C. 1 min, 50° C. 1 min, and 72° C. 1 min) using HD sense primer (5′ AAAAGGATCCATGGCGACCCTGGAAAAG 3′, containing a BamHI site (SEQ ID NO: 7)) and c-myc antisense primer (5′ AGATCCTCTTCTGAGATGA 3′ (SEQ ID NO: 8)). PCR products were treated with proteinase K (1 mg/ml final concentration) for 30 min at 37° C. before being gel purified using QIAEX II kit (QIAGEN). Purified HD-Q25 and Q104 PCR products were then digested with XmaI and BamHI restriction enzymes and ligated separately into a pGEX vector. HD-Q25 and Q104 PCR products were also digested with BamHI and SmaI restriction enzymes and ligated separately into pQBi25c3 vector (QUANTUM Biotechnologies, Montreal, Quebec, Canada) digested with BamHI and NruI.

The plasmid pQBi-25c3-HD-Q(−) was prepared by digestion of pQBi-25c3-HD-Q104 with EcoRI and HindIII. After inactivation of the enzymes at 75° C. for 10 min, the digested products were treated with Klenow polymerase in presence of dNTPs at 30° C. for 30 min to generate blunt-ended DNA. The digested vector was then gel purified and self-ligated.

The plasmid pGEX-HD-42Q and HD-65Q were prepared as followed: cDNA segments coding for 42Q and 65Q were isolated from pBSKS-42Q and pBSKS-65Q by digestion with HindIII and XmaI. After gel purification, cDNA segments were cloned into pGEX-HD 25Q which was digested with the same restriction enzymes to replace the HD-25Q cDNA coding sequence. The relevant amino acid sequences of the expressed experimental polypeptides of the invention are summarized in below in Table 3.

TABLE 3
Amino Acid Sequence of Experimental Polypeptides

| | |
|---|---|
| Protein name | Sequence |

Bacterial expression

| | |
|---|---|
| GST-DRPLA-Q35 | GST-lvprgsVSTHHHHH(Q)₃₅HHG |
| (SEQ ID NO: 9) | NSGPPefpgrlerphrd |
| GSTHD-Q25 | GST-lvprgsMATLEKLMKAFESLKS |
| (SEQ ID NO: 10) | F(Q)₂₅lqpgstraaas |
| GST-HD-Q42 | GST-lvprgsMATLEKLMKAFESLKS |
| (SEQ ID NO: 11) | F(Q)₄₂lqpgstraaas |

Mammalian expression

| HD-Qn-GFP | MATLEKLMKAFESLKSF(Q)ₙ-GFP |
|---|---|
| (SEQ ID NO: 12; n = 47) | |
| (SEQ ID NO: 13; n = 72) | |
| (SEQ ID NO: 14; n = 104) | |
| HD-Qn-Myc-HIS₆ | MATLEKLMKAFESLKSF(Q)ₙlqpgg |
| (SEQ ID NO: 15; n = 47) | stmsrgpfeqkliseedln |
| (SEQ ID NO: 16; n = 72) | mhtehhhhhh |
| (SEQ ID NO: 17; n = 104)) | |
| GFP-HD-Q25 | GFP-idgggggkgpvtgtgsMATLEK |
| (SEQ ID NO: 18) | LMKAFESLKSF(Q)₂₅lqpriltn |
| GFP-HD-Q104 | GFP-idgggggkgpvtgtgsMATLEK |
| (SEQ ID NO: 19) | LMKAFESLKSF(Q)₁₀₄lqpriltn |
| GFP-DRPLA-Q81 | GFP-idgggggkgpvtgtgsVSTHHH |
| (SEQ ID NO: 20) | HH(Q)₈₁HHSGPPef |
| Abbreviations: | |
| DRPLA, Dentatorubral-pallidoluysian atrophy (atrophin-1); HD, Huntington disease (huntingtin); GST, glutathione transferase; GFP, green fluorescent protein. The atrophin-1 and huntingtin protein sequences are underlined. The flanking sequences are part of the expression vectors. The polypeptide sequences of GST and GFP are art recognized, see, e.g., GFP: plasmid pQB125-fC3 (cat# AFP2133) FROM quantum Biotechnologies, 1801 de Maisonneuve Bvld. West Montreal, Quebec, h3H 159, CANADA; | |
| #GST: plasmid pGEX-4T-3 (cat# 27-4583-01) from PHARMACIA (Genbank Accession #U13855), and the GST-DRPLA constructs described in Onodera, et al., (1996) Toxicity of expanded polyglutamine-domain proteins in Escherichia coli. | |

Protein Expression and Purification

V_{H}-linker-V_{L} cDNA coding sequences from N-HD-C4 sFv was excised from the pHEN-1 vector by digestion with NcoI and NotI restriction enzymes. After gel purification, the cDNA was ligated into a pSYN1 vector already digested with the same restriction enzymes. Ligated plasmids were then introduced into DH5α competent bacteria by electroporation and amplified plasmid DNA was purified with QIA prep spin miniprep kit (QIAGEN Inc., Valencia, Calif.). The N-HD-C4 sFv was expressed in E. coli strain TG1 as followed: transformants were inoculated into 10 ml of 2×TY medium containing 100 ug/ml ampicillin and 1% glucose. After overnight incubation at 37° C., the culture was then inoculated into 1 liter of 2×TY medium containing 100 ug/ml ampicillin and 0.1% glucose and agitated vigorously at 37° C. until an Abs₆₀₀ₙₘ=1 was achieved. IPTG was then added to 0.1 nM final concentration and the culture was incubated overnight at RT and at 200 rpm. Cells were harvested by centrifugation and suspended in 30 ml of PBS at pH 8.0. Lysozyme was added to 1 mg/ml final concentration and the mixture was incubated for 5 min at RT. The bacterial lysate was then sonicated on ice to shear the chromosomal DNA and centrifuged at 25,000×g for 30 min at 4° C. to remove bacterial debris and other insoluble material. The supernatant was incubated with 1 ml pre-washed Ni-NTA resin (QIAGEN) in PBS containing 20 mM imidazole for 2 hours at 4° C. The resin was then washed with 30 ml of PBS, followed by 10 ml of PBS containing 35 mM imidazole and 10 ml of PBS containing 40 mM imidazole. The sFv molecules were then eluted with PBS containing 250 mM imidazole in 1 ml fractions. Fractions of interest were pooled and dialyzed in PBS.

GST-fusion proteins were expressed in E. coli strain BL21 as follows: fresh transformants were inoculated into 1 liter LB medium containing 100 μg/ml of ampicillin and 0.1% glucose and agitated vigorously at 37° C. until an Abs₆₀₀ₙₘ=1. IPTG was then added to 0.1 nM final concentration and the culture was incubated for 3 h and 200 rpm. Cells were harvested by centrifugation and suspended in 30 ml of PBS at pH 8.0. Lysozyme was added to 1 mg/ml final concentration and the mixture was incubated for 5 min at RT. The bacterial lysate was sonicated and centrifuged at 25,000×g for 30 min at 4° C. The supernatant was incubated with 1 ml pre-washed glutathione-Sepharose 4B beads (Pharmacia) in PBS for 2 hours at 4° C. The resin was then washed with 50 ml of PBS and proteins were eluted with 50 mM Tris at pH 8.0 containing 10 mM reduced glutathione in I ml fractions. Fractions of interest were pooled and dialyzed in PBS.

Immunopurification Experiments

About 3 ug of N-HD-C4 sFv was mixed with 4 ug of either GST-DRPLA-Q35 or GST-HD-Q25 or GST-HD-Q42 in PBS (200 μl final volume) and rocked for 1 h at RT. Twenty microliters of glutathione Sepharose 4B beads (50% slurry, pre-washed with PBS) was added and incubated for another 30 min at RT to capture the sFv/GST fusion protein complexes. The beads were harvested by centrifugation at 12,000×g for 10 sec and washed thrice with 1 ml PBS. The complexes were eluted with 10 μl of 50 mM Tris pH 8.0 containing 10 mM reduced glutathione for 10 min at RT. Tubes were then centrifuged at 12,000×g for 1 min and the supernatants containing the soluble complexes were transferred to new tubes, mixed with an equal volume of 2× SDS-sample buffer and heated at 95° C. before being loaded on SDS-PAGE.

Co-Transfection of COS 7 Cells and Immunofluorescence

COS 7 cells were grown on cover glass in 6-well plates (105 cells/well) and co-transfected at a ratio of 3:1 (sFv to target) with plasmids containing either N-HD-C4 sFv or a negative control sFv (containing an HA tag and SV40 nuclear localization sequence (NLS)) and plasmids containing GFP-targeted fusion proteins using 5 μl SuperFect transfection reagent (QIAGEN). At 24 hours post-transfection, cells were prepared for immunofluorescence as follows: cells were fixed with 4% paraformaldehyde and permeabilized with 0.2% triton-X100. To prevent non-specific binding of secondary antibodies, cells were blocked with 10% normal goat serum in 3% BSA. Then, pre-absorbed polyclonal rabbit anti-HA IgG was added to detect sFv antibodies and bound IgG was revealed by rhodamine conjugated goat anti-rabbit IgG antibodies (PIERCE).

Intrabodies

Retargeting intrabodies were constructed as follows. Briefly, the plasmid pcDNA3.1 (+) (Invitrogen, Carlsbad, Calif.) was modified to encode an influenza hemagglutinin epitope (YPYDVPDYA (SEQ ID NO: 43)), representing the HA immunotag and either a SV40 nuclear targeting sequence (TPPLLLRLV (SEQ ID NO: 44)), a lysosomal targeting signal, or no targeting signal (the absence of a targeting signal leaves the sFv in the cytoplasm) and the C4 sFv specific for huntingtin (anti-HD C4) or irrelevant sFv controls. The expression of the sFv intrabody constructs was under the control of the cytomegalovirus (CMV) promoter.

Kinetic Binding Analysis

The Kd of purified scFv clone C4 was determined by using surface plasmon resonance in a BIAcore 2000 (Biacore AG, Uppsala, Sweden). In a BIAcore flow cell, approximately 50 resonance units (RU) of biotinylated HD-peptide (250 nM in 1M NaCl and 50 mM NaOH) was coupled to a streptavidin sensor chip, pre-conditioned with three consecutive 1-minute injections of 1 M NaCl in 50 mM NaOH. This amount of coupled peptide resulted in a maximum of 40 to 80 RU of bound intrabody. For regeneration of the surface after binding of intrabody, 5 μl of 50 mM NaOH containing 1 M NaCl was injected, resulting in a return to baseline. Association was measured under a continuous flow of 5 μl/min with a concentration range from 60 to 100 nM, The kₒₙ was determined from a plot of In (dR/dt)/t versus concentration, where R is response and t is time (Karlsson et al., J. Immunol. Methods 145: 229-240 (1991)). The k_{off} was determined from the dissociation part of the sensorgram at the highest concentration of intrabody analyzed by using a flow rate of 20 μl/min. The Kd was calculated as k_{off}/kₒₙ.

Brain Tissue Cultures

Organotypic slice cultures are made from P12-P14 cerebellum, cerebral cortex, or striatum, using published methods with minor modifications (1)(14). The brain is dissected from a mouse and placed in a beaker of high magnesium (1.8 mM CaCl₂ and 2.4 mM MgCl2) HEPES-buffered Hanks' saline (HBHS) at 4° C. The brain is then blocked and mounted next to a 2% agarose block on a 1-cm² piece of Plexiglas (0.3 cm thick) with SuperGlue®. Dental wax mounts the plastic onto the slicing-chamber tissue-holder. After being mounted, the tissue is placed in the slicing chamber of the vibratome. The bath of the vibratome contains cooled high magnesium HBHS at 4° C. Temperature is maintained by a peltier thermoelectric refrigeration system attached to the slicing chamber (FHC, Brunswick, Me.). Three or 400 μm thick sections were sliced from the tissues of interest. As each new slice was made it was placed into growth medium at 4° C. until the collection of slices was complete. Slices from one preparation were then placed in an enclosed filter unit, and incubated at 37° C. for 1 hour in high magnesium artificial cerebral spinal fluid (12 mM NaCl, 0.33 mM KCl, 0.12 mM NaH₂PO₄, 2.5 mM NaHCO₃, 1 mM dextrose, 2.4 mM MgCl₂, 1.8 mM CaCl₂; all items from Sigma®, St. Louis, Mo.). Solutions were constantly gassed with 95% O₂ and 5% CO₂. Slices, grown at the air-media interface, were maintained on 0.4 μm Transwell® filters (Coming Costar®, Cambridge, Mass.). The growth medium contains 25% heat inactivated horse serum, 25% HBHS, 50% MEM without bicarbonate, 4 mM L-glutamine, 30 mM D-glucose, 50 mM sodium bicarbonate, and 12.5 mM HEPES (all items from Sigma, St. Louis, Mo.). Temperature was maintained at 33° C. and 5% CO₂. Media was changed three times a week.

Transfection of Brain Tissue Cultures

Plasmids were introduced into neuronal tissue using a Biolistic PDS-1000/He Particle Delivery System, a “gene gun”, from BIO-RAD. The plasmids were coated on the microcarriers, which are composed of gold or tungsten. The microcarriers were then loaded onto a macrocarrier. The macrocarrier was accelerated by high pressure helium and a partial vacuum towards the stopping screen. The stopping screen halts the macrocarrier but allows the microcarriers to continue towards and penetrate the target cells. Organotypic cultures were then placed in petri dishes containing 1% agar for bombardment, after which the culture the were returned to the growth chamber. Typically, sections between 300 and 400-μm thick were used.

Microscopy

Standard inverted and upright fluorescent microscopy was used. In addition, however, laser confocal microscopy may be applied as previously described (see e.g., Becker et al., IEEE Transactions on Biomed Eng. 45:105-118 (1998); Turner et al., Neuron 4:833-845 (1990); and Turner et al., International Rev. Exp Path 36:53-72 (1996)).

Single-Chain Fv-Phage Library Construction and Selection of Human sFv Antibodies Specific to the Amino-Terminal HD Peptide

Single-chain Fv library construction from spleens of mice immunized against an N-terminal huntingtin peptide having an altered number of glutamine residues and fused to GFP may be performed as follows. Briefly, cDNA corresponding to the V_{H} and V_{L} genes can be made from total spleen RNA of immunized mice using Superscript II RNase H reverse transcriptase (GIBCO BRL) and primers to the junction between the variable and constant regions of gamma heavy chains and kappa light chains, respectively. The V_{H} and V_{L} genes are, e.g., amplified by 25 cycles of PCR (94° C. for 1 min, 55° C. for 1 min and 72° C. for 2 min) from the cDNA with Pfu polymerase (Stratagene) and VH primers [VH1 BACK (34) and VH1 FOR2 (49), and VL primers VK2BACK and VK4FOR (10)], respectively. The linker DNA may be prepared by mixing an equimolar ratio of the following primers, using Klenow polymerase and deoxynucleotide triphosphates (dNTPs) for the fill in reaction.

| | |
|---|---|
| LinkerBack: | |
| (5′-TCACCGTCTCCTCAGGTGGAGGCGGTTCAG | (SEQ ID NO: 21)) |
| GCGGAGGTGGCTCT | |
| LinkerFor: | |
| (5′-TGGGTGAGCTCATGTCCGMTCCGCCACCGC | (SEQ ID NO: 22)) |
| CAGAGCCACCTCCGCCTG | |

The V_{H} and V_{L} PCR products and the double stranded linker DNA are then purified on an agarose gel and 1 μg of each V_{H} and V_{L} product and 300 ng of DNA linker were mixed in a 50 μl PCR reaction mix without primers. After 7 cycles (94 C for 2 min and 72 C for 4 min), 25 pmol each of VH1 BACK-SfiI (VH1 BACK with a SfiI restriction site) and VK4FOR-NotI (VK4FOR with a NotI restriction site) primers are added to the reaction mix and amplified for 20 cycles (94 C for 1.5 min and 72 C for 2.5 min). After gel purification the assembled V_{H}-linker-V_{L} PCR products are extensively digested with SfiI and NotI and cloned into the pHEN1 vector. The ligation mixture is electroporated in small aliquots into TG1 competent cells. The diversity of the library may be analyzed by PCR screening of recombinant colonies with VH1BACK and VK4FOR primers, followed by digestion with BstNI restriction enzyme (Gussow, et al., Nucleic Acid Res. 17:4000 (1989)). The phage library can then be amplified by scraping the colonies into 50 ml 2×TY medium containing 100 mg/ml ampicillin and 1% glucose, shaken for 45 min at 37° C., and the phage rescued by the addition of helper phage.

Biotin-labeled antigens can be prepared, using, e.g., NHS-Biotin (Pierce Chemical Co.), which non-specifically reacts with e-amino groups or lysine residues. For a given library, an aliquot may be equilibrated with its corresponding antigen. Streptavidin-coated magnetic beads are then added and the streptavidin binds the antigen-phage antibody complexes, which are isolated by magnetic separation of the beads from the library solution; the beads are then washed. Bound sFv-phage is eluted from the beads with acid solution, pH 2, immediately neutralized, and rescued by infecting bacteria. Rescued phage can be amplified and used for another round of selection.

Selection may be done using, e.g., fusion proteins expressed in COS-7 cells from the original constructs, which have a combination of flanking epitopes specific to HD and polyglutamine repeat lengths. The latter can be identified by a second selection against heterologous polyglutamine proteins (e.g., using DRPLA), and the former by cross-reactivity with an isoform of the protein that does not contain more than 25 copies of glutamine (Gln).

As described in Example 2, four rounds of selection were performed with decreasing concentrations of antigen at each round (100 nM, 50 nM, 10 nM, 1 nM). After the third round, bacterial clones were screened to isolate those which secreted sFv molecules specific to the N-terminal HD sequence. Screening was performed by ELISA. Bacterial supernatants containing secreted sFv were incubated with either 2% bovine serum albumin (BSA), as negative control, or with 2% BSA containing biotinylated antigen (0.1 μg/well, final concentration) in pre-blocked microtiter plates overnight at 4° C. The mixtures were then transferred to streptavidin-coated (0.1 μg/well, final concentration) microtiter plates. After an overnight incubation, plates were washed and mouse anti-myc IgG (9E10, 0.1 μg/well final concentration) was added. Bound IgG was detected with alkaline phosphatase conjugated to goat anti-mouse IgG.

For further characterization, it was necessary to purify soluble sFv proteins. Therefore, the cDNAs coding for V_{H}-linker-V_{L} of the selected clones were transferred from pHEN to pSYN-1 vector. The pSYN-1 vector allows protein expression with a 6×His tail, which facilitates rapid purification on Ni-NTA columns. The binding specificity of these sFv molecules can be tested by affinity purification as follows:

About 3 μg of each clone were incubated in solution for 1 hour at room temperature with 4 ug of either GST-DRPLA-Q35, GST-HD-Q25 or GST-HD-Q42. Then, a 10 μl bed volume of glutathione-Sepharose 4B (Pharmacia) was added to each tube to precipitate the GST-fusion protein/sFv complexes. After 30 min incubation, tubes were centrifuged and the pellets washed 3 times with 1 ml PBS. The complexes were eluted from glutathione-Sepharose beads by addition of 10 mM reduced glutathione to the pellets.

After centrifugation, supernatants containing the eluted complexes were transferred to new tubes and mixed with an equal volume of 2× SDS-loading buffer. The samples were boiled and the proteins separated on a 12% SDS-PAGE. Protein bands were visualized by Coomassie blue staining, and/or transferred to nitrocellulose membranes in order to detect the sFv molecules by immunoblot.

### Example 1

### Methods for Assaying the Effects of Intracellular Polypeptide Aggregation

In this example, methods for assaying intracellular polypeptide aggregation and a demonstration of intracellular polypeptide aggregation leading to cell death is provided.

In order to determine the effects of intracellular polypeptide aggregation, cells expressing a polypeptide representing normal huntingtin polypeptide as compared to cells expressing a huntingtin polypeptide representing the polypeptide found in patients with Huntington's disease was examined. Primate cells (COS-7) were transfected with plasmid constructs encoding a model huntingtin-GFP fusion polypeptide representing the normal polypeptide or an altered huntingtin polypeptide associated with disease having 47, 72, or 104 glutamine residues. Within 24 h of transfection, a large number of the cells express the huntingtin polypeptide as indicated by the GFP tag fused to each of the test proteins. Observations were recorded at three time points (see Table 4).

TABLE 4

| | huntingtin polypeptides having increasing | | | |
|---|---|---|---|---|
| | amounts of glutamine residues | | | |
| | | | 72Q | |
| Time | 25Q (normal) | 47Q (mutant) | (mutant) | 104Q (mutant) |
| 24 h | diffuse, bright | diffuse, bright | some small, | light soma with |
| | | | bright | brilliant |
| | | | aggregates | aggregates |
| 48 h | diffuse, bright | some foci of | many with | huge aggregates |
| | | brighter label | brilliant | and some cell |
| | | | aggregates | death |
| 72 h | diffuse, bright | some brilliant, | many large | substantial death |
| | | small, | aggregates | |
| | | aggregates | | |

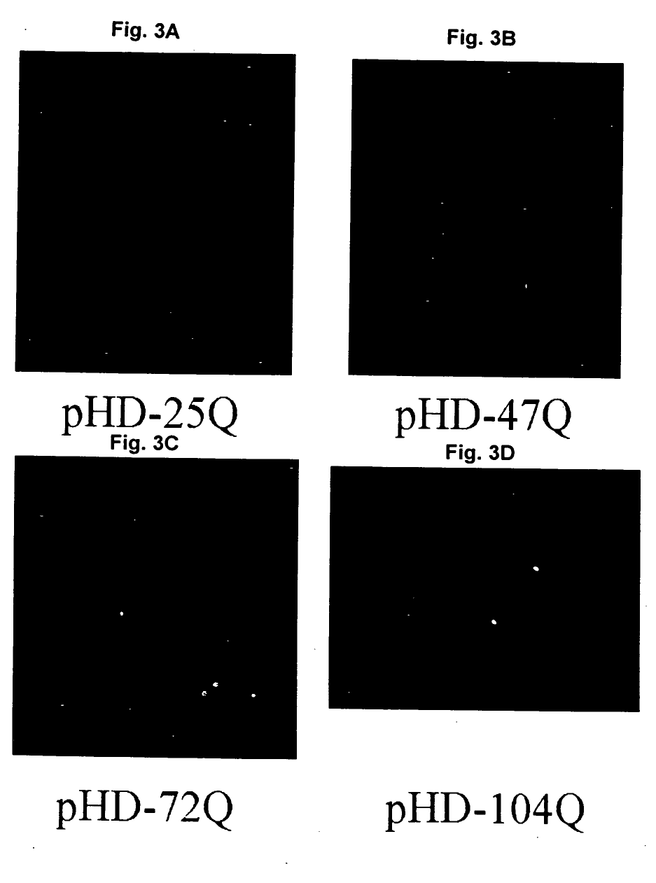

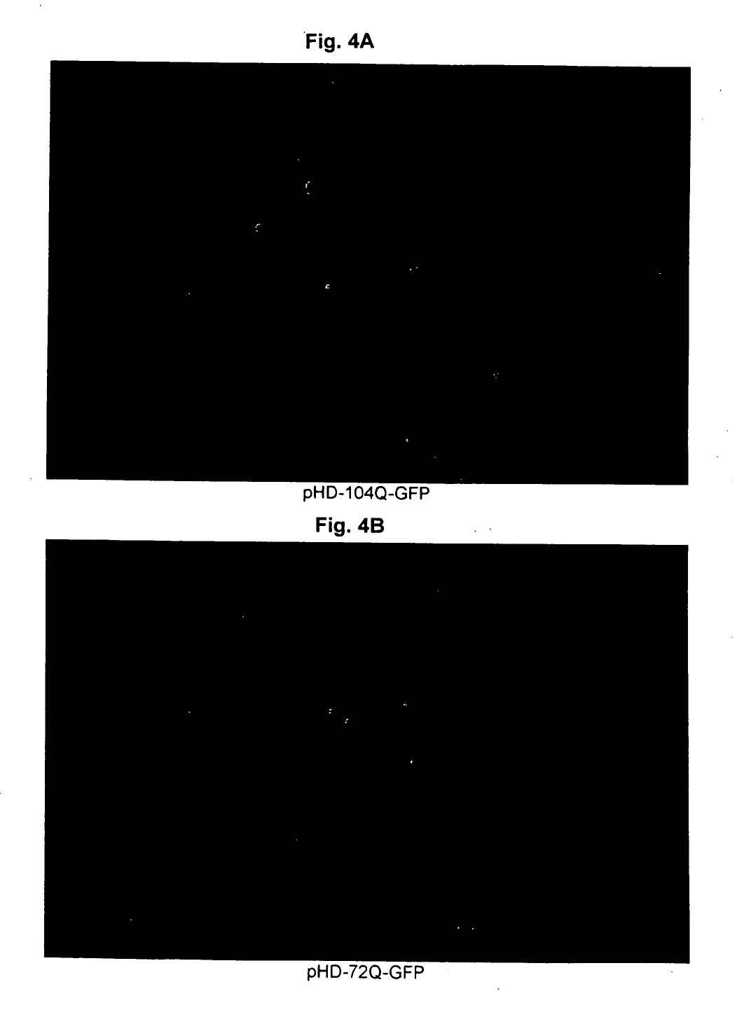

Examples of the 48 h time point are shown in FIG. 3. Using fluorescence microscopy, it was observed that cells that express the huntingtin-GFP polypeptide with 104 glutamine residues (FIG. 3D) harbor large, brilliant aggregates. The highest amount of cell death was observed in these cells. In cells expressing a model huntingtin polypeptide with fewer glutamine residues (72Q), extremely bright and more variable aggregates are seen, with slightly less cell death. And in cells expressing a model huntingtin polypeptide with 47 glutamine residues, the fluorescence intensity was less and more diffuse. Cells expressing a model huntingtin polypeptide with a normal range of glutamine residues (25) exhibited a diffuse fluorescence with no evidence of polypeptide aggregation.

Thus, this experiment demonstrates a concordance between altered huntingtin polypeptides and intracellular aggregation and cell death. Accordingly, this assay system recapitulates in vitro, a range of Huntington's disease pathologies and can be used for screening therapeutics that alter intracellular polypeptide aggregation and polypeptide aggregate-mediated cell death (see Example 2).

### Example 2

### Methods for Engineering and Selecting Intrabodies with Binding Specificity to a Neuronal Polypeptide

In this example, methods for identifying and selecting an intrabody with affinity for a selected polypeptide are presented.

In order to generate an intrabody capable of inhibiting the formation of an intracellular polypeptide aggregate comprising the huntingtin polypeptide, a biotinylated peptide corresponding to the 17 N-terminal amino acid residues of huntingtin (Nt-HD) was generated as an antigen to capture phage displaying sFv molecules specific to this sequence. Briefly, a human sFv-phage display library containing 10⁹ different clones, was incubated with biotinylated Nt-HD using a peptide synthesized at the Protein Core Facility, Tufts University (Boston, Mass.). Streptavidin-coated magnetic beads were then added and the streptavidin complexed with associated sFv-phage antibodies were isolated and washed. Bound sFv-phage were eluted with acid, neutralized, and rescued by infecting bacteria. Rescued phage were then amplified and used for another round of selection.

Four rounds of selection were performed with decreasing concentrations of antigen at each round (i.e., 100 nM, 50 nM, 10 nM, 1 nM). After the third round, bacterial clones were screened to isolate those which secreted sFv molecules specific to the Nt-HD sequence. Screening was performed using ELISA. Ninety clones were screened and 20 clones showed higher binding to the biotinylated Nt-HD peptide (at least ten times above that of the negative controls). In order to test the binding specificity of the sFv species 8 clones were selected, which gave an OD of 0.2 on ELISA, out of the 20 clones for larger-scale sFv expression. Bacterial supernatants containing sFv molecules were assayed by ELISA on microtiter plates coated with streptavidin, GST-HD-42Q, GST-HD-65Q, and GST-DRPLA-35Q (as a negative control, from Onodera et al. (33)). For the positive control, bacterial supernatants were incubated in solution with biotinylated N-terminal HD peptide before transfer to microtiter plates coated with streptavidin. Bound sFv was detected with α-9E10 IgG followed by alkaline phosphatase conjugated goat anti-mouse IgG. From this experiment, one clone (designated as cc-Nt-HD-C4 sFv) was identified that reacted preferentially with antigens containing the Nt-HD flanking peptide sequence.

For further characterization, it was necessary to purify soluble sFv proteins. Therefore, the cDNAs coding for V_{H}-linker-V_{L} of the selected clones were transferred from the pHEN-1 vector to the pSYN-1 vector. The pSYN-1 vector allows for protein expression and periplasmic secretion of the sFv fused to a hexaHis tail, which facilitates rapid purification. Accordingly, two subclones of α-Nt-HD-C4 sFv were expressed in bacteria and resultant polypeptides were purified on Ni-NTA columns (Qiagen®).

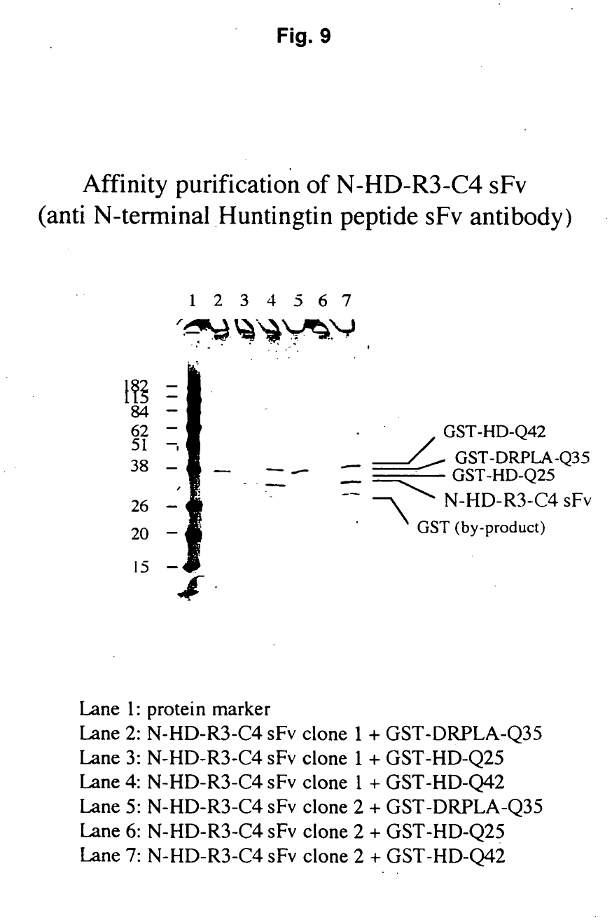

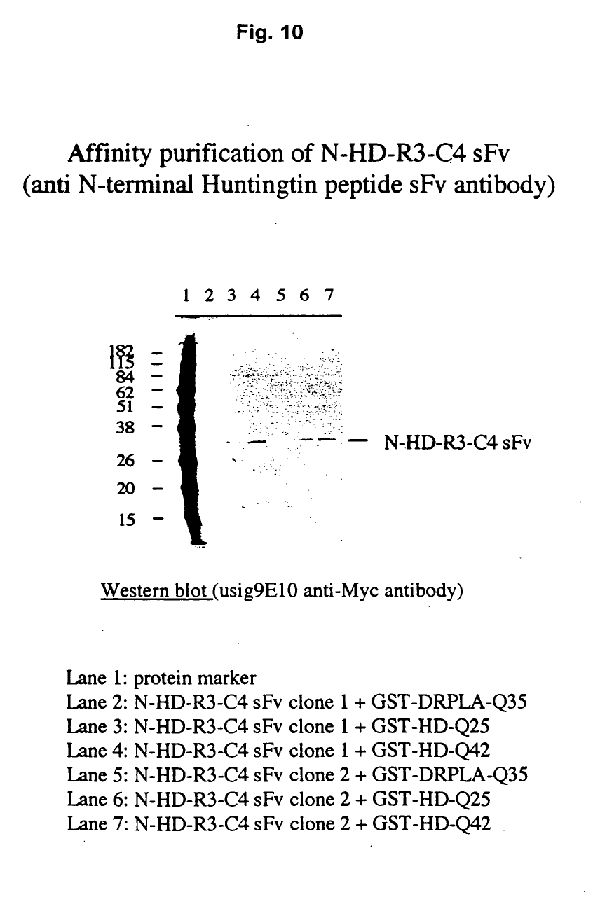

The binding specificities of the selected sFv clones were tested by affinity binding to a full range of antigens, as follows: (a) using sample numbers that correspond to the experimental results in FIGS. 9 and 10, about 3 μg of α-Nt-HD-C4 sFv clone 1 (lanes 2-4) or α-Nt-HD-C4 sFv clone 2 (lanes 5-7) were mixed with 4 μg of the following antigens and incubated in solution for 1 hr at room temperature (lanes 2 and 5) GST-DRPLA-35Q, (lanes 3 and 6) GST-HD-25Q, (4 and 7) GST-HD-42Q; (b) a 10 μl bed volume of glutathione-Sepharose 4B (Pharmacia) was added to each tube to bind all GST-containing protein complexes; the beads were washed with 10 mM glutathione solution, which eluted the GST-antigen or the sFv-antigen complex, and these eluates were analyzed by SDS-PAGE. Protein bands were visualized by Coomassie blue staining (FIG. 9) and were also transferred to nitrocellulose membranes in order to detect the sFv molecules by immunoblot blot (FIG. 10). Both subclones showed the desired specificity of binding only the HD flanking peptide.

For example, FIG. 9 shows that only in samples containing the Nt-HD sequence (i.e. GST-HD-25Q and GST-HD-42Q), a band corresponding to α-Nt-HD-C4 sFv is present at about the same intensity as the other proteins. Immunoblot analysis also confirmed that both anti-C4 sFv clones were present in samples containing the N-terminal HD sequence but were not detected in samples containing GST-DRPLA-Q35 (FIG. 10).

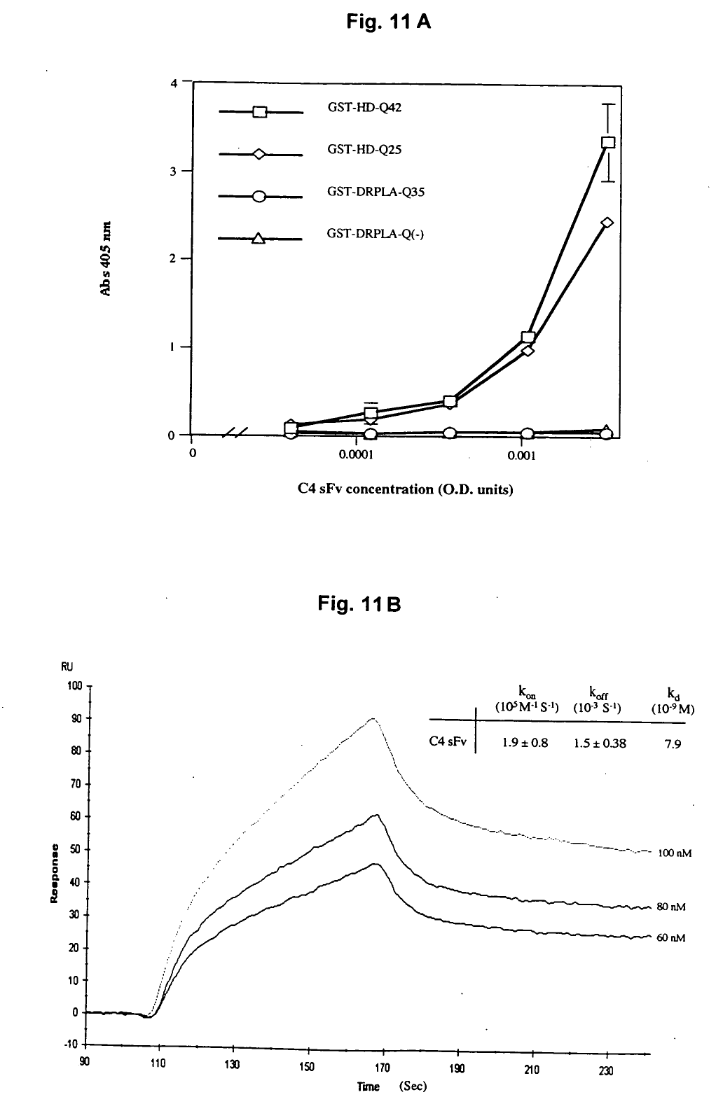

To characterize the precise binding properties of the intrabody of the invention, further qualitative and quantitative studies of sFv binding to immobilized antigen were conducted in vitro. The C4 sFv antigen binding properties were monitored by ELISA assays that likewise confirmed that the C4 sFv binds specifically to the N-terminal HD sequence (FIG. 11A). For quantitative binding studies, the HD peptide (1-17) with C-terminal biotin was loaded at very low concentrations onto a streptavidin-coated BIAcore sensor chip, in order that kinetics measurements were made under conditions where mass transport artifacts are negligible. The kinetics of association and dissociation were determined for the C4 sFv antibody in the BIAcore 2000 (FIG. 11B). The Kd was calculated to be 7.9 nM, based on the measured ka and kd kinetic constants using a 1:1 Langmuir model for simple bimolecular interactions (BIAevaluation 3.0 software, Biacore International AB, Uppsala, Sweden) (FIG. 11B, insert upper right). The corresponding binding affinity (Ka) of 1.3×10⁸ M⁻¹ is typical of antibodies selected to rigid antigens from this large phage display library and indicates this is a rather high affinity sFv, especially against a short peptide sequence (Sheets et al. (1998) Proc. Natl. Acad. Sci. USA 95: 6157-6162). These results are suggestive that the HD (1-17) peptide may form a relatively rigid epitope that is recognized by the C4 sFv combining site rather than a wide range of unstructured conformers that would significantly reduce the apparent binding constant.

These results demonstrate the ability to identify and select α-Nt-HD-C4 sFv intrabody molecules that are specific to a particular epitope such as the N-terminal sequence of huntingtin. Accordingly, an intrabody that detects any specific N-terminal or C-terminal epitope unique to a selected polypeptide may be selected. Moreover, using the above method, an intrabody specific to a polyglutamine-rich domain can be generated. Such an intrabody could bind to any polypeptide of interest having a polyglutamine-rich region, for example, huntingtin, Ataxin , Atrophin, etc. Finally, these methods afford the engineering of a multivalent intrabody having dual or even multiple specificities for a selected polypeptide. A multivalent intrabody would be capable of binding, e.g., the target polyglutamine-rich region of a target polypeptide as well as other N- and/or C-terminal flanking epitopes of the polypeptide.

### Example 3

### Methods for Inhibiting Polypeptide Aggregation in Mammalian Cells Expressing a Pathological Huntingtin Polypeptide

In this example, methods for inhibiting the formation of aggregates and retargeting intracellular polypeptides in mammalian cells are presented.

In order to demonstrate the ability of an intrabody to specifically bind an intracellular polypeptide and inhibit polypeptide aggregation, mammalian cells (COS-7) were cotransfected with a first plasmid encoding a model huntingtin polypeptide fused to GFP and a second plasmid encoding an intrabody that specifically binds to the model huntingtin polypeptide. The assay is designed such that both the target antigen, i.e., the model huntingtin polypeptide, and the intrabody can be visualized. In particular, the huntingtin polypeptide is visualized by detecting fluorescence emitted by the GFP domain of the huntingtin-GFP fusion polypeptide and the intrabodies tested are visualized using a rhodamine-conjugated antibody that can specifically bind to the intrabody.

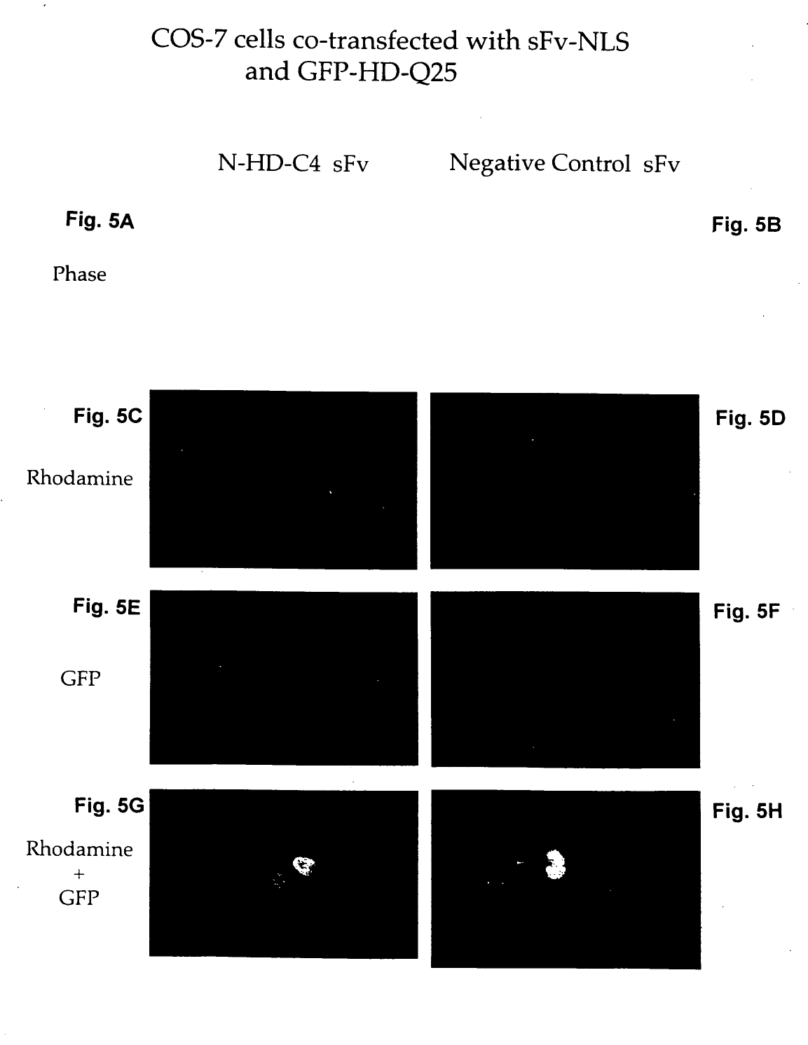

Accordingly, experimental results presented in FIGS. 5A-H show, using fluorescence microscopy, the ability of an intrabody to specifically bind and retarget a huntingtin polypeptide in a cell. In FIGS. 5A and 5B, the distribution of cells is first shown using phase contrast light microscopy. In FIGS. 5C, 5E, and 5G it was observed that cells coexpressing a model huntingtin-GFP fusion polypeptide (HD-25Q-GFP) (FIG. 5E) and an intrabody that specifically binds a model huntingtin polypeptide that further comprises a nuclear targeting signal (α-Nt-HD-C4 sFv-NLS), can retarget the distribution of huntingtin polypeptide to the nucleus as demonstrated by a confluence of rhodamine and GFP staining in the nucleus (FIG. 5G). In contrast, cells expressing an intrabody that binds an unrelated protein, fail to retarget the distribution of huntingtin polypeptide (FIGS. 5D, 5F, and FH).

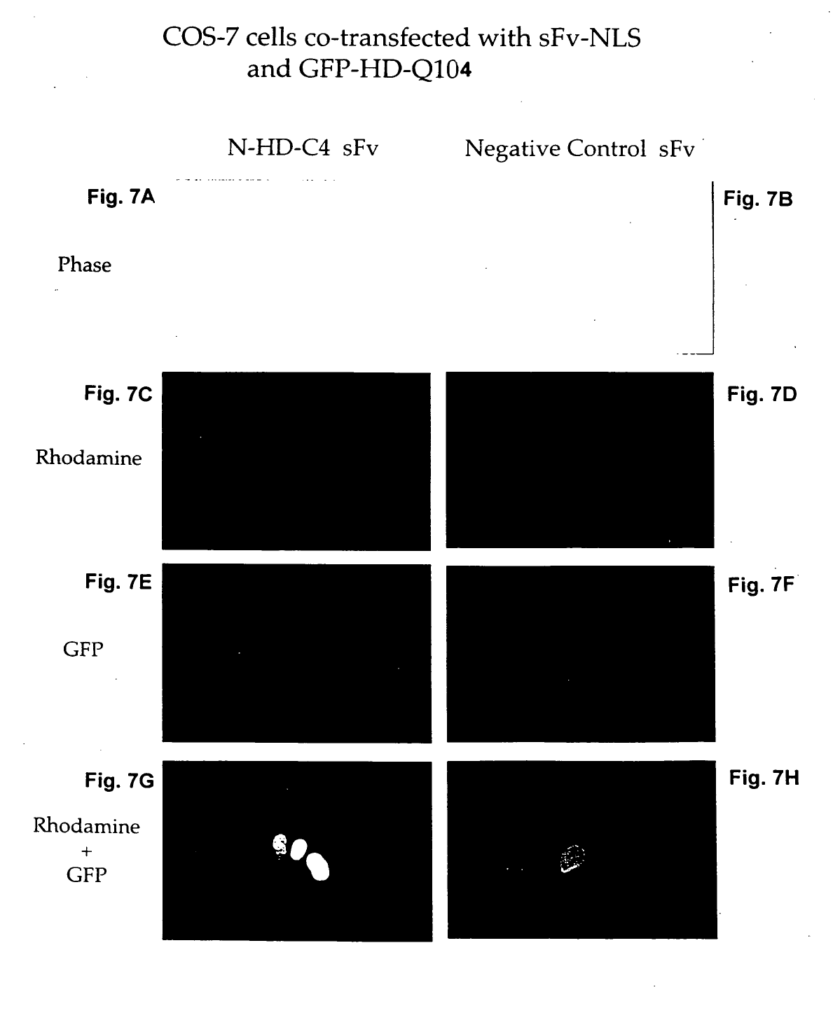

These experiments were extended to demonstrate that the intrabody tested above would also have the same efficacy on an altered huntingtin polypeptide having a polyglutamine repeat that is associated with Huntington's disease and has the highest propensity to form intracellular aggregates. Thus, mammalian cells were transfected with plasmids encoding the model altered huntingtin polypeptide with 104 polyglutamines (pHD-104Q-GFP) and a second plasmid encoding either a huntingtin specific intrabody (α-Nt-HD-C4 sFv) or an intrabody having binding specificity to an irrelevant polypeptide. FIGS. 7A-H show, using fluorescence microscopy, that only the huntingtin specific intrabody can specifically retarget the cellular distribution of the model altered huntingtin polypeptide (i.e., having 104 glutamine repeats) to the nucleus (FIGS. 7C, 7E, and 7G) whereas the irrelevant intrabody has no effect (FIGS. 7D, 7F, and 7H).

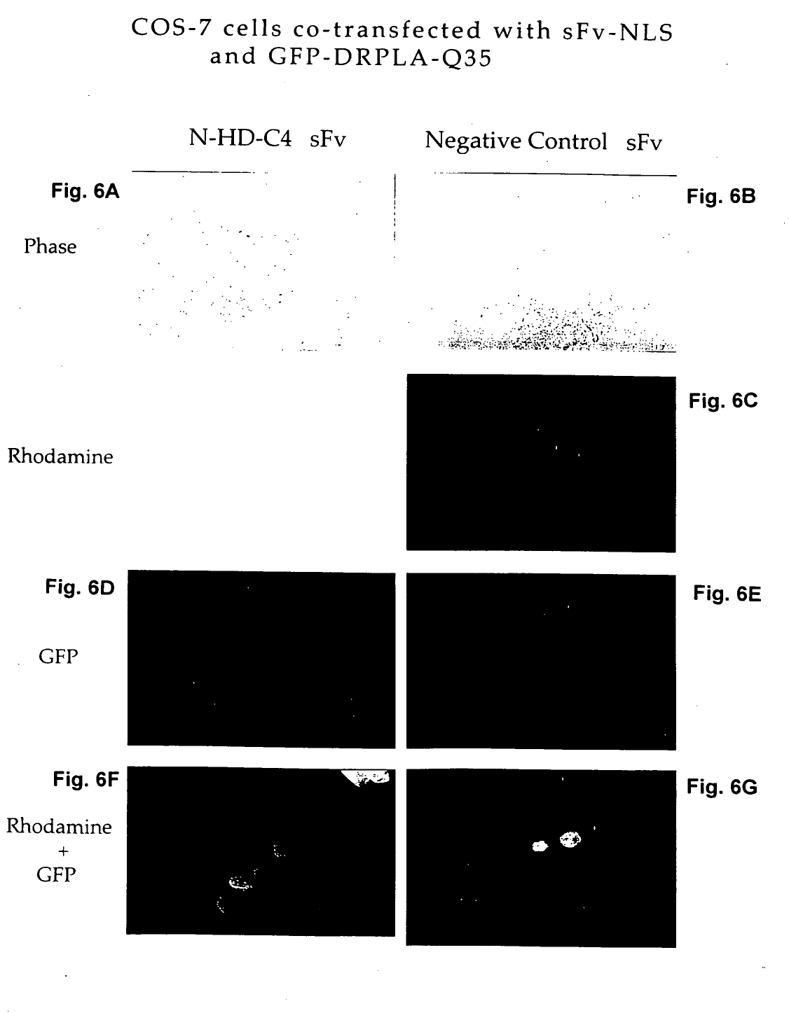

To further demonstrate the specificity of the huntingtin specific intrabody (α-Nt-HD-C4 sFv), mammalian cells were cotransfected with another target polypeptide that is polyglutamine rich and can aggregate and cause neurological disease (DRPLA). The model DRPLA target polypeptide was fused to a GFP domain to track the expression and intracellular localization of the target polypeptide in the transfected cells. In addition, two different intrabodies, one specific to huntingtin, and a second intrabody specific to an unrelated polypeptide, were independently cotransfected with the DRPLA target polypeptide. Cells (COS-7) coexpressing the glutamine rich DRPLA polypeptide (GFP-DRPLA-35Q) (FIGS. 6C-G) and an intrabody against either huntingtin polypeptide (α-Nt-HD-C4 sFv; FIGS. 6D, 6F) or another unrelated polypeptide (Negative Control sFv; FIGS. 6C, 6E, and 6G) show no change in the cellular distribution of DRPLA polypeptide.

In addition, experiments were performed that established that the huntingtin specific intrabody can prevent the formation of intracellular polypeptide aggregation as well as retarget the undesired polypeptide to a different cellular location. Mammalian cells were transfected with plasmids encoding altered huntingtin polypeptides having the highest polyglutamine content (72 or 104 glutamine residues) and greatest tendency to aggregate and induce disease, with one of the huntingtin-specific intrabodies having either no targeting signal, a nuclear targeting signal, or a lysosomal targeting signal.

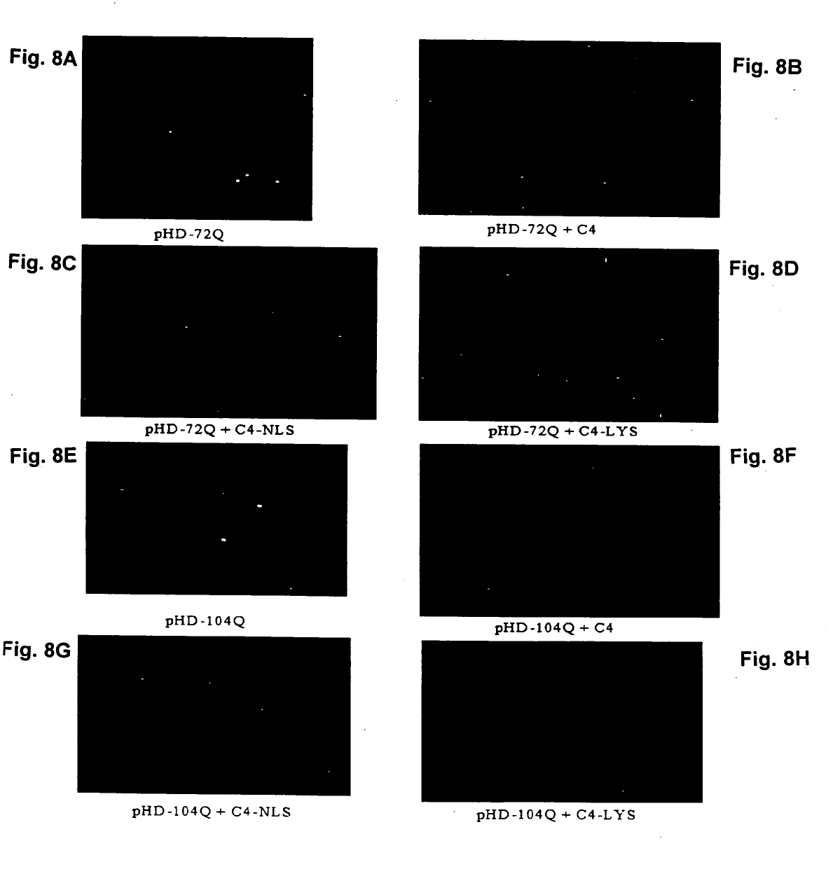

Each of the three intrabodies tested, using fluorescence microscopy, was shown to inhibit the formation of intracellular polypeptide aggregates (8B-8D, 8F-8H) as compared to controls (FIG. 8A, 8E). This is evidenced by a reduction in the bright GFP signals emitted by the polypeptide aggregates. In addition, it was also demonstrated that retargeting of the model huntingtin polypeptide was achieved if contacted with an intrabody having either a nuclear targeting signal (see FIGS. 8C, 8G), or a lysosomal targeting signal (FIGS. 8D, 8G). This was indicated by a accumulation of GFP signal in the nucleus of those cells expressing an intrabody with a nuclear targeting signal (FIGS. 8C, 8G). In cells transfected with an intrabody with a lysosomal targeting signal, a more diffuse signal throughout the cytoplasm was observed (FIGS. 8D, 8H).

Still further, in yet another test of the ability of the intrabody of the invention to inhibit the formation of intracellular aggregates of HD, the anti-HD C4 sFv intrabody and HD-polyQ-GFP were cotransfected into several different cell types and a statistical analysis of intrabody-mediated inhibition of polypeptide aggregation was measured.

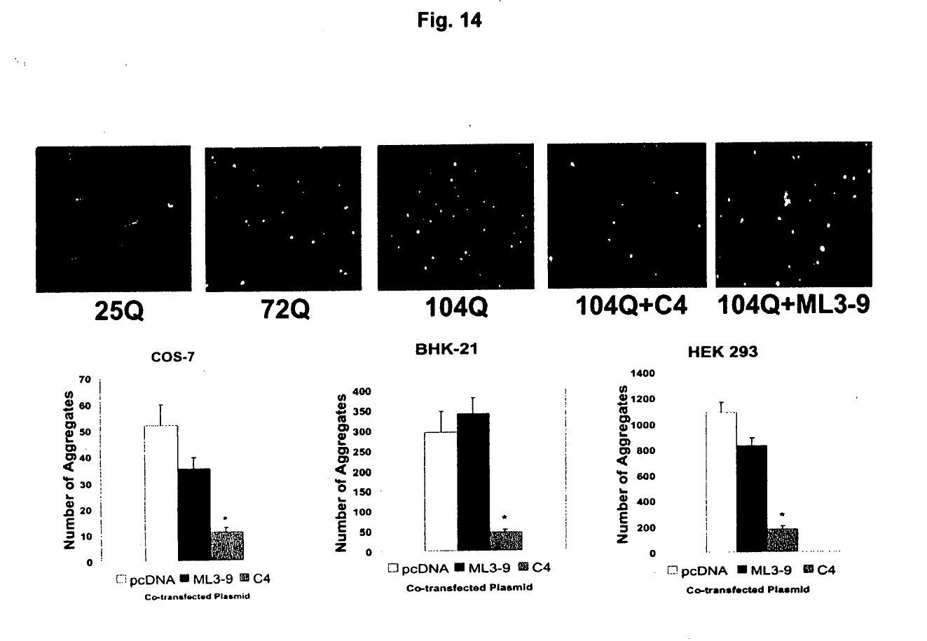

In particular, it was observed that the formation of pathological HD aggregates in situ display a striking punctate fluorescent morphology with polyglutamine lengths of 40 or more residues (i.e., with Q72 or Q104, but not Q25; FIG. 14). In addition, because these polypeptide aggregates are detergent-insoluble, a method for improved quantitation of residual fluorescent bodies after SDS lysis of transfected cells could be performed (Kazantsev et al., PNAS 96:11404-11409 (1999)). This improved assay allows for polypeptide aggregates to be more accurately enumerated because the plane of focus is eliminated as a variable in the experiment. Accordingly, this assay was utilized for quantitation of polyQ antigen aggregation, alone or in the presence of C4 sFv or control intrabody. COS-7, BHK, and 293 cells were co-transfected with plasmids that express the C4 or a control intrabody and HD-polyQ-eGFP fusions containing Q25, Q72, or Q104. Using HD-polyQ72-eGFP or HD-polyQ104-eGFP, a qualitative decrease in the extent of aggregation at an intrabody to antigen plasmid ratio of 3:1 was observed, but the most significant quantitative effects were found at a ratio of 5:1 (FIG. 14). In contrast to the pronounced reduction in aggregation seen with C4 sFv, control transfections with irrelevant intrabody constructs (C8, or ML3-9 sFv) showed no statistically significant reduction in aggregate formation compared with the parent empty vector. Cells transfected with the Q25 plasmid alone never showed SDS-insoluble aggregates.

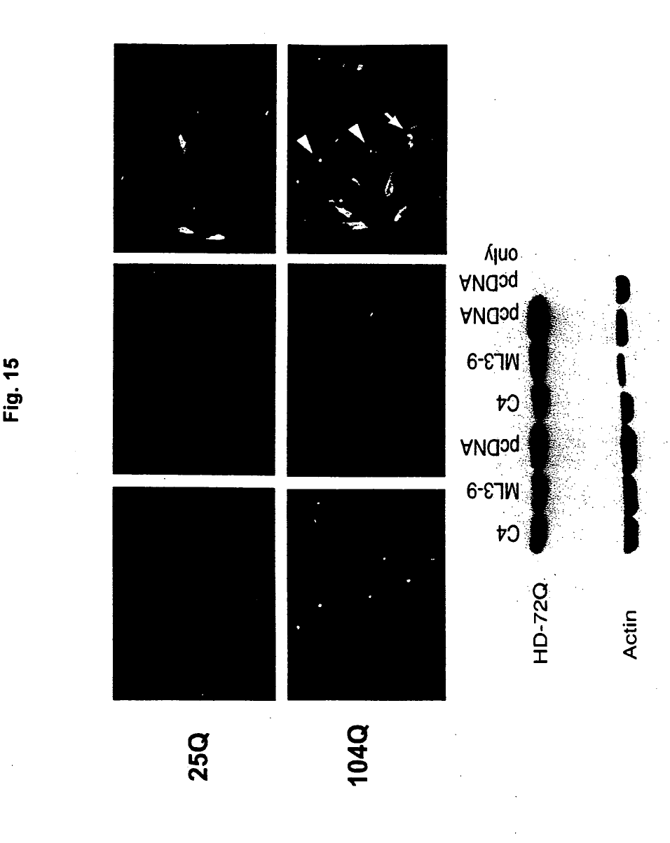

The typical intrabody constructs were fused to a C-terminal hemagglutinin (HA) peptide tag to facilitate immunodetection of the protein, while keeping the protein localized to the cytosol. Double label immunofluorescence studies confirmed that BHK cells expressing HD-polyQ72-GFP or HD-Q104-GFP (green label, FIG. 15) plus anti-HD C4 sFv (red label, FIG. 15) showed substantial diffuse GFP label, but with only an occasional fluorescent aggregate (usually labeled with HA as well; note arrows in FIG. 15). Those cells that did not appear to be expressing the intrabody showed evidence of large aggregates (FIG. 15, arrowheads, upper panel). The diffuse labeling was also characteristic of the normal length (25Q) construct, with or without intrabody co-transfection (FIG. 15). The reduced aggregate formation observed with dual transfectants did not appear to derive from suppression of HD protein expression, since immunoblots showed indistinguishable levels of products in the different transfection paradigms (FIG. 15, lower panel).

Accordingly, these results confirm the ability of an intrabody to specifically bind to and inhibit the formation of polypeptide aggregates associated with a neurological disease (e.g., Huntington's Disease). In addition, these results also demonstrate the ability to retarget an undesired intracellular polypeptide using an intrabody with a targeting signal.

### Example 4

### Methods for Analyzing the Intracellular Specificity of an Intrabody within Mammalian Cells

In this example, methods for analyzing the specificity of intrabodies in mammalian cells expressing a targeted antigen are presented.

In order to demonstrate the specificity of the anti-huntingtin intrabody (α-Nt-HD-C4 sFv), mammalian cells (COS-7) were cotransfected with a model HD target polypeptide and with either α-Nt-HD-C4 sFv or with an intrabody specific to an unrelated polypeptide. The model HD target polypeptide was fused to c-myc (EQKLISEEDL (SEQ ID NO: 45)) epitope tag (HD-Q104-myc). The intrabodies sequences were fused to a HA tag and a SV40 nuclear localization signal (sFv-HA-NLS), so that bound antigens should be retargeted to the cell nuclei. At 48 hours post-transfection, cells were fixed and permeabilized. The HD-Q104-myc proteins were detectable using anti-myc epitope antibodies (i.e., MAb 9E10 IgG), followed by FITC-labeled goat anti-mouse IgG. The sFv-HA-NLS intrabodies were detected by polyclonal rabbit anti-HA IgG followed by rhodamine-conjugated goat anti-rabbit IgG antibodies.

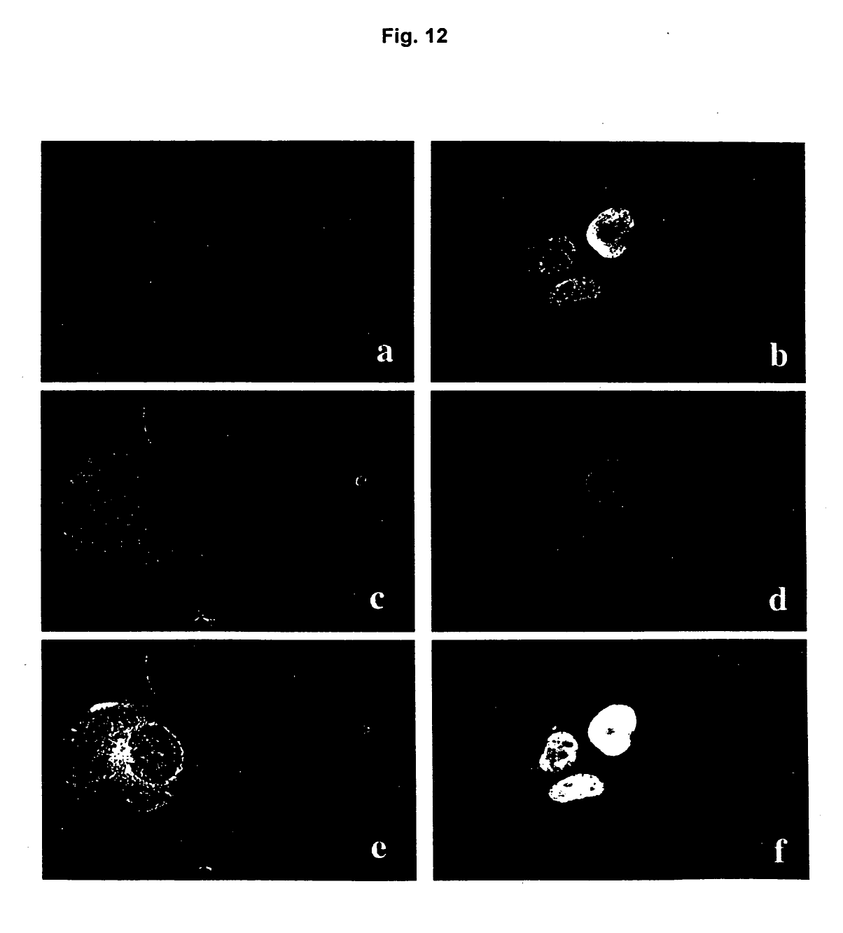

Experimental results presented in FIGS. 12A-F show that although both intrabodies are located in the cell nuclei FIG. 12A-B), only α-Nt-HD-C4 sFv-HA-NLS is able to relocate the model altered huntingtin polypeptide HD-Q104-myc from the cytosol to the nucleus (FIGS. 12D, 12F versus 12C, 12E, respectively). Additionally, the control intrabody did not prevent aggregation of the HD-Q104 antigen (shown by arrowheads in panels C and E).

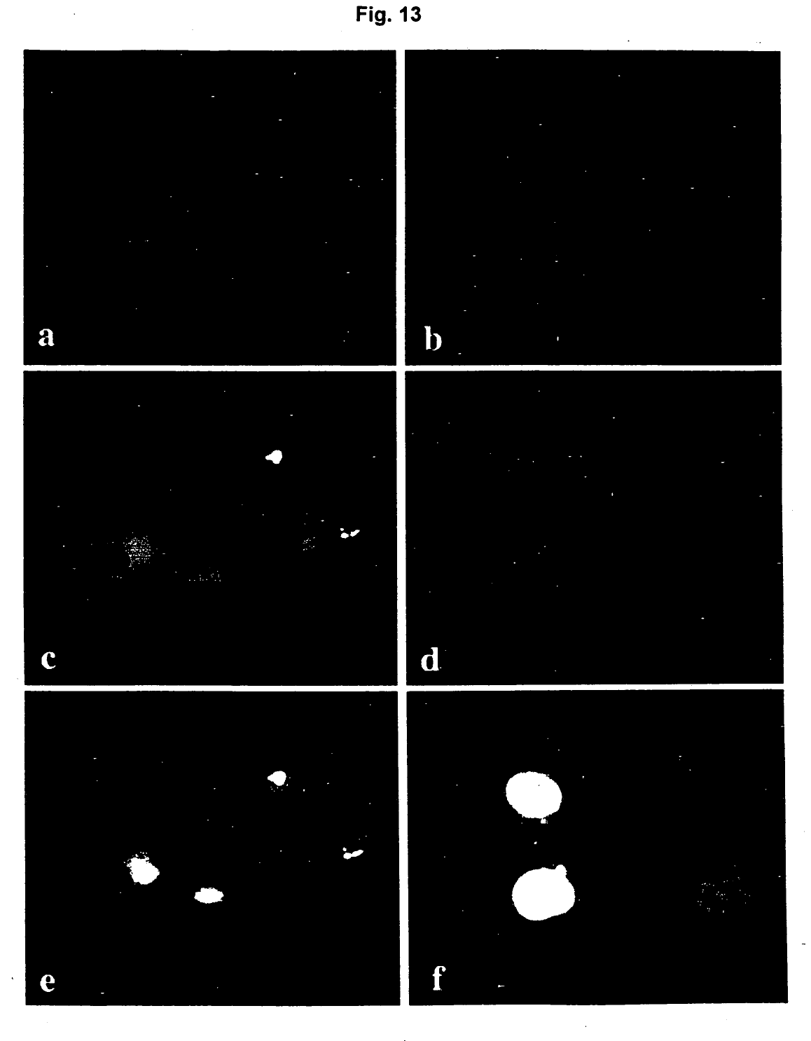

Another method for demonstrating the specificity of intrabodies for a targeted antigen intracellularly, is to analyze cells that coexpress the specific intrabody with either the targeted antigen or with an irrelevant antigen. FIGS. 13A-F show stably transfected COS-7 cells expressing α-Nt-HD-C4 sFv-HA-NLS which were further transfected with plasmids encoding either the model altered huntingtin polypeptide with 104 glutamine repeats (FIGS. 13B, D, F) or the model altered DRPLA polypeptide with 81 glutamine repeats (FIGS. 13A, C, E). Both model altered polypeptides were fused to GFP (GFP-HD-Q104 and GFP-DRPLA-Q81, respectively). The anti-Nt-HD-C4 sFv-HA-NLS intrabody clearly shows specificity to GFP-HD-Q104 (FIGS. 13D, F) but not to GFP-DRPLA-Q81 (FIGS. 13C, E) since only the GFP-HD-Q104 colocalized with the intrabody inside the cell nuclei.

These methods allow rapid evaluation of intrabody specificity in a relevant intracellular environment and are applicable to not only the antigens discussed herein, but may also be used to test other intrabody/targeted antigen pairs.

### Example 5

### Methods for Assaying Polypeptide Aggregation in Mammalian Brain Tissue

In this example, methods for detecting the ability of a mutant huntingtin polypeptide to from intracellular aggregates in mammalian brain are demonstrated.

In order to demonstrate that altered huntingtin polypeptide can form intracellular aggregates in the mammalian brain, cerebellar slices from mice (P12 Balb) were biolistically transfected with plasmids encoding polypeptides representing normal and altered huntingtin polypeptides using a gene gun, and examined after 24 h. When plasmids encoding model altered huntingtin-GFP polypeptides having 104 glutamine residues (HD-104Q-GFP) were transfected into a 24-hr-old culture, large aggregates, indicated by brilliant points of fluorescence, were observed throughout the brain tissue. Similarly, when plasmids encoding altered huntingtin GFP polypeptides having 72 glutamine residues (HD-72Q-GFP) were transfected into a 24-hr-old culture, smaller, more discrete aggregates, indicated by brilliant points of fluorescence, where observed.

In addition, this assay also affords the ability to identify particular cell types expressing a model huntingtin polypeptide. For example, Purkinje cells can be identified using an antibody to calbindin and neurons can be visualized using an antibody against neurofilaments. Accordingly, by using a red label to visualize these cell-specific antibodies, the identification can be superimposed on cells that are positive for the GFP portion of the HD-polyQ-GFP fusion protein. The slices are then examined using laser confocal microscopy.

Importantly, using, e.g., the methods presented in Example 2, this assay can be adapted to assay the ability of intrabodies to inhibit the formation of intracellular polypeptide aggregation (e.g., of a huntingtin polypeptide) or retarget such polypeptides in brain tissue with multiple cell types. In addition, this assay affords that ability to screen for other binding molecules that can disrupt the formation of polypeptide aggregates. Finally, this assay may also be used to isolate particular neuronal cell types (e.g., striatal cells) for long term culture and for assaying as described herein.

### Example 6

Methods for Generating an sFv Phage Library to a Neuronal-Polypeptide

In this example, methods for generating an intrabody phage library to a selected antigen using DNA vaccination is demonstrated.

In order to be able to generate intrabodies specific to a wide range of possible polypeptides, a DNA vaccination strategy was developed. This method has the advantage of generating a library “directed” to a particular epitope rather that selecting for a rare intrabody in a “naïve” library. Accordingly, the number of clones that must be screened is greatly reduced. Instead of protein, DNA was used to immunize mice, so that the resultant phage display libraries can be made from spleens activated to express antibodies to epitopes in their intracellular or extracellular conformations, rather than epitopes only to forms that the protein can take in vitro.

Plasmid constructs that expressed fusion proteins comprising the first 17 amino acids of huntingtin, varying lengths of polyglutamine repeat, and a modified green fluorescent protein were used to express antigen in vivo in eight female mice (BalbCBy/J genetic background). Plasmid constructs encoding a model huntingtin N-terminus fused to GFP and having either a normal number of glutamine residues or an altered number of residues associated with Huntington's disease, were injected into mice in a vehicle of normal saline at a concentration of 0.5 μg/μl.

Mice were bled prior to immunization for pre-immune serum. All mice were injected with one of the plasmid constructs on day 1 (50 μg of plasmid DNA intramuscularly and 50 μg of plasmid DNA intradermally—100 μg total). Injections were repeated on day 14 and the mice were bled on day 21. Mouse sera was then screened for a positive humoral response by probing immunoblots of pHD-25Q transfected COS7 cell lysates with varying dilutions of serum. The stimulated spleens of positive responder mice were determined to be candidates for the generation of monoclonal antibodies, intrabodies, and phage display.

Of the eight mice injected, 50% (4 of 8) demonstrated a positive antibody response capable of detecting a fusion protein in an immunoblot assay with a minimal serum dilution of 1:100 (see Table 5). Of these, 75% (3 of 4) were immunized with the 104 glutamine repeat construct.

TABLE 5

| Immunizing | | | |
|---|---|---|---|
| Construct | Mouse | Positive Response | Maximal Dilution |
| pHD14-104Q | 1 | Yes | 1:200 |
| pHD14-104Q | 2 | No | |
| pHD17-25Q | 3 | No | |
| pHD17-25Q | 4 | No | |
| pHD14-104Q | 5 | Yes | 1:1500 |
| pHD14-104Q | 6 | Yes | 1:500 |
| pHD17-25Q | 7 | Yes | 1:500 |
| PHD17-25Q | 8 | No | |

Thus, these results demonstrate that, using the methods described herein, an antigen specific intrabody binding domain can be engineered using the above source material and, e.g., a phage display library to generate a specific antibody binding molecule. Moreover, these methods can be readily applied to virtually any other antigen of interest.

### Example 7

### Methods for Testing Intrabody Inhibition of a Neurological Disease in a Mammal

In this example, methods for testing the ability of an intrabody to treat or cure a neurological disease in a living mammal are presented.

In order to determine the ability of an intrabody to inhibit the formation of altered neuronal polypeptide aggregates associated with neurological disease in a living mammal, a method using transgenic mice has been developed.

In particular, animal models for Huntington's disease and SCA have been developed (see, e.g., Table 2).

For example, colonies of transgenic mice genetically engineered to express an altered huntingtin polypeptide have been established (as described by Bates et al. ((3)(29)) and these animals exhibit several neurological hallmarks of Huntington's disease. Mice are symptomatic, and begin to show neuronal huntingtin polypeptide aggregates in cortical, striatal, and cerebellar Purkinje cells by 8 weeks of age.

In addition, a second animal model representing a SCA neurological disease caused by altered ataxin expression have been established. These animals have an expanded-repeat ataxin gene that is expressed under the control of a Purkinje cell-specific promoter. These animals show symptoms by 12 weeks, and the first histopathology in Purkinje cells by 4 weeks.

In yet a third approach, an animal model representing DRPLA caused by altered Atrophin-1 can be established (Schilling et al., Neuron 24:275-286 (1999)).

Accordingly, these animal represent three in vivo assay systems in which to test the ability of an intrabody to inhibit the formation of neuronal polypeptide aggregates and thus prevent, treat, or delay the onset of disease. For example, the intrabody may be delivered intracranial as a polypeptide or as an expressible nucleic acid construct using any of the methods described herein.

In addition, in yet another way to demonstrate the ability of an intrabody to prevent, treat, or delay the onset of a neurological disease, these animals can be crossed against animals expressing an intrabody that can be conditionally expressed. Thus, at different points during the clinical course of the disease, the intrabody may be tested for its in vivo efficacy. In particular, the Tet regulatory system of Nestler and colleagues can be employed which involves two transgenic mice lines, one containing the Tet-regulated Transactivator (tTA) under the control of a neuron-specific enolase (NSE) promoter which directs expression in neuronal tissues, and one containing a construct with a gene of interest, for example, either the anti-huntingtin intrabody (i.e., α-Nt-HD-C4 sFv) or an anti-ataxin intrabody cloned downstream of the Tet-regulated promoter (TetOp) (Chen et al., Mol Pharmacol. 54:495-503 (1998); Arnold et al., PNAS 94:8842-8847 (1997); and Clark et al., J. of Neurosci. 17:7325-7395 (1997)). To activate expression of a selected intrabody, doxycycline (a Tet derivative) is removed from the drinking water of progeny carrying the intrabody gene and altered neuronal polypeptide (e.g., either huntingtin or ataxin).

The breeding scheme is as follows: first, a bigenic mouse line carrying both the NSE-tTA and HD transgenes are generated where the presence of both transgenes is determined by PCR of genomic DNA, and the expression of the genes is verified by immunoblots. These mice can be used initially as a source of brain slice cultures that are permissive for the expression of the TetOp-intra-C4 plasmid in neurons, and later bred to the TetOp transgenic mice.

Using NSE-tTA brain slices, both HD-polyQ and TetOp-intra-C4 can be transfected to assay whether the expressed intrabodies function to reduce the number of aggregates formed (e.g., by targeting the aggregates for lysosomal degradation). The successful plasmids can then be injected to create transgenic founder mice. These can be bred for two generations, and tested for uninduced levels of intrabody.

To completely inhibit transgene expression, doxycycline can be continuously administered to animals at the low dose of 25 ug/ml (Chen et al., Mol Pharmacol. 54:495-503 (1998)). At this low dose, intrabody induction upon doxycycline removal can be rapid. To address at what stage of disease progression is the intrabody therapy most effective, doxycycline can be removed from drinking water of various age groups of HD-NSE-tTA x TetOp-intra-C4 mice at 4 weeks, 6 weeks, 8 weeks, and 10 weeks. Brains from these animals can be collected and assayed for presence and number of aggregates as compared to its wild type sibs as an indication of the effectiveness of the intrabody therapy.

As noted above, these experiments can be used to test an anti-HD intrabody with HD transgenic mice, and an anti-ataxin intrabody with SCA1 mice, and an anti-polyglutamine intrabody with both strains of mice. The bigenic NSE-tTA/HD or ataxin mice can be used for both the specific and the more general intrabody breeding.

Accordingly, this method allows for an in vivo determination of the most effective of the intrabodies identified in the in vitro assays. In addition, these methods allow for a determination of whether the intrabody will reduce aggregate toxicity to levels necessary to observe behavioral rescue of neurological symptoms in HD (or SCA1) mice. Finally, these methods also allow for a determination of what stage of disease progression at which the induction of the intrabody expression is most effective.

### Example 8

### In Vivo Demonstration of a Nucleic Acid Vaccine for Eliciting a Therapeutic Host Antibody Immune Response Against Undesired Intracellular Polypeptide Complexes

In this example, methods and compositions for eliciting intrabodies by a host against undesired intracellular polypeptide aggregates, are presented.

In order to determine the ability of an intrabody made by the immune system, instead of being administered exogenously, to inhibit the formation of altered neuronal polypeptide aggregates associated with neurological disease, test animals were immunized with a DNA vaccine encoding a polypeptide antigen representative of a pathogenic intracellular polypeptide, i.e., huntingtin polypeptide. The DNA vaccine encoding the test polypeptide, as compared to a control, provoked a therapeutic humoral response in immunized animals resulting in the formation of host intrabodies (i.e., antibodies) against the DNA encoded huntingtin immunogen as well as endogenous intracellular huntingtin polypeptide.

In brief, the experimental approach was as follows. Test animals (HDR6/2 transgenic mouse) that express a fragment of mutant human huntingtin containing approximately 165 glutamines (normal is less than 35 copies) were obtained. These mice develop subtle behavioral and neurochemical changes measurable by 5 weeks of age, with severe neurological disease apparent at 11-12 weeks. Survival beyond 15 weeks is rare. Importantly, these mice also develop a huntingtin-associated diabetes syndrome similar to that seen in humans, as evidenced by increased fasting serum glucose levels (at 10 weeks of age). Huntingtin-associated diabetes has been attributed to abnormal accumulations of mutant HD protein in the islet cells of the pancreas.

For this experiment, mice were immunized to produce antibodies to part of the HD protein fragment that was used to select intrabodies in the previous experiments presented herein. In particular, HDR6/2 mice were immunized at 5 weeks of age, and boosted at 7 weeks, with 100 μg injections of a plasmid expressing the HD transgene fragment linked to Green Fluorescent Protein (GFP). Two injections were done at each age, one into leg muscle and the other intradermally just below the tail. Mice were bled at 8 weeks to determine responsiveness, using immunoblots made from lysates of HEK-293 cells 24 hours after transfection with the original injection plasmids.

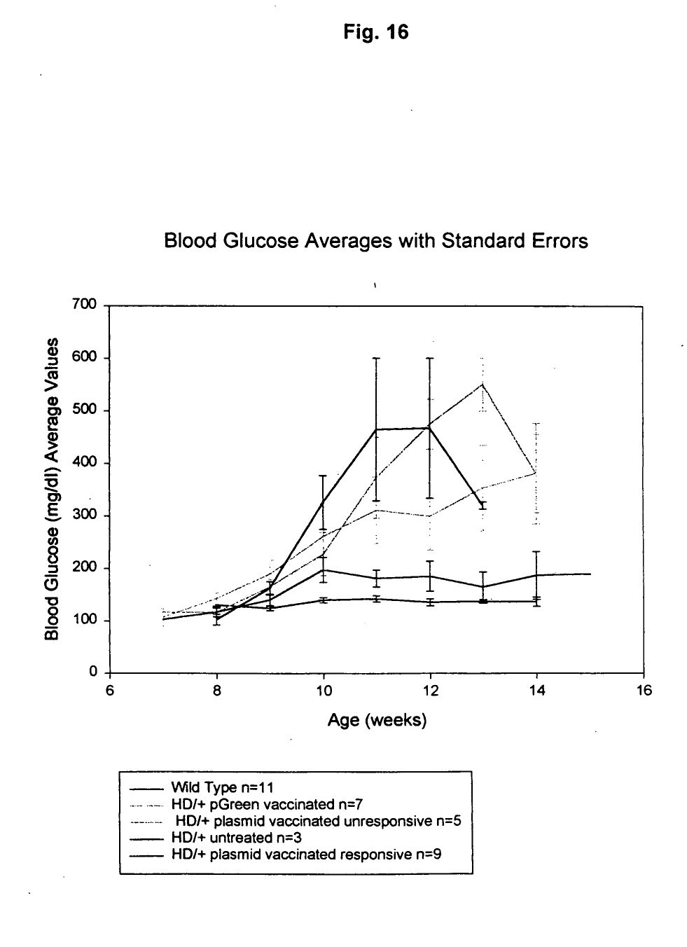

Blood glucose levels were then compared for those HD mice that showed a positive immune response versus those that did not respond. Blood glucose was determined in duplicate using an Advantage Glucometer with Comfort Curve test strips, after a 6 hour fast. The fasting blood glucose levels of the untreated transgenic HD mice were significantly higher than those of wild-type mice starting at 10 weeks of age. Transgenic mice that did not respond to the plasmid immunization, or those that were immunized with a plasmid containing only the GFP sequence, showed levels in the range of the untreated mice. However, mice that were plasmid immunized and responsive as assayed by immunoblot, had significantly reduced blood glucose levels, completely within the range of the wild-type mice at ages 13 and 14 weeks. Thus, HD immunization can prevent development of the pancreatic phenotype (see FIG. 16).

In addition to glucose levels, pancreatic levels of mRNA coding for insulin were also measured. A series of mice was sacrificed at 14 weeks, and RNA was extracted from the pancreas of each. Samples were pooled based on genotype, treatment and response. Insulin mRNA levels were determined by reverse transcription polymerase chain reaction (RT-PCR). Samples from wild-type mice showed an intense band at the location predicted based on the primers used in the RT-PCR reaction. Untreated and non-responding mice showed only a faint band of the same size, demonstrating a defect in insulin biosynthesis. Those mice that had been immunized and responsive showed a band representing insulin mRNA that was close to wild-type in intensity as confirmed by densitometry. Thus, the mechanism of the therapeutic improvement appears to be restoration of insulin mRNA levels.

Finally, sera from mice that responded to DNA vaccination, was tested to determine if the animals generated antibodies that bound to the polypeptide antigen used to select the therapeutic intrabody discussed above. Accordingly, a peptide corresponding to the first 17 amino acids of huntingtin was synthesized and immobilized on a test dish. Then, an ELISA test of epitopes present in positive responders was performed: mouse serum that was positive by immunoblot, and that elicited both a reduction in abnormal glucose levels and an increase in insulin mRNA, also showed binding to the 17 amino acid peptide.

In summary, these findings are consistent with antibodies produced by immunization having the capacity to act intracellularly to prevent or reverse pathogenesis due to mutant polypeptide function due to, e.g., aggregation or accumulation.

### REFERENCES CITED IN THE SPECIFICATION

1. F. Owen, et al., Lancet, 51 (1989); K. Hsiao, et al., Nature 338, 342 (1989); K. Hsiao, et al., Science 250, 1587 (1990) ; K. Hsiao, et al., Nature Genet. 1, 68 (1992).
2. A. M. Goate, et al., Nature 349, 704 (1991), D. Gaines, et al., ibid. 373, 523 (1995); N. Suzuki, et al., Science 264, 1336 (1994).
3. R. Sherrington, et al., Nature 375, 754 (1995); D. Scheuner, et al., Nature Med. 2, 864 (1996); K. Duff, et al., Nature 383, 710 (1996); D. Borchelt, et al., Neuron 17, 1005 (1996); M. Citron, et al., Nature Med. 3, 67 (1997); L. Holcomb, et al., ibid. 4, 97 (1998).
4. E. Levey-Lahad, et al., Science 269, 973 (1995); D. Scheuner, et al., Nature Med. 2, 864 (1996); F. Oyama, et al., J Neurochem. 71, 313 (1998).
5. M. Polymeropoulos, et al., Science 276, 2045 (1997).
6. T. Gasser, et al., Nature Genet. 18, 262 (1998) .
7. M. Farrer, et al., Hum. Mol. Genet., 8:1901-1905 (1999).
8. M. G. Spillantini, et al., Brain Pathol. 8, 387 (1998); P. Poorkaj, et al., Ann. Neurol. 43, 815 (1998); M. Hutton, et al., Nature 393, 702 (1998); M. G. Spillantini, et al., Proc. Natl. Acad. Sci. U.S.A. 95, 7737 (1998).
9. J. Brown, et al., Hum. Mol. Genet. 4, 1625 (1995).
10. D. R. Rosen, et al., Nature 362, 59 (1993); M. E. Gurney, et al., Science 264, 1772 (1994); N. Shibata, et al., J. Neuropathol. Exp. Neurol. 55, 481 (1996).
11. A. R. La Spada,.et al., Nature 352, 77 (1991); A. Abdullah, et al., Hum. Mol. Genet. 7, 379 (1998); P. M. Bingham, et al., Nature Genet. 9, 191 (1995); M. Li, et al., Ann. Neurol. 44, 249 (1998).
12. The Huntington's Disease Collaborative Research Group, Cell 72, 971 (1993); S. W. Davies, et al., ibid. 90, 537 (1997); E. Scherzing et al., ibid., p. 549.
13. R. Koide, et al., Nature Genet. 6, 9 (1998); S. Nagafuchi et al., ibid., p. 14; M. W. Becher et al., Neurobiol. Dis. 4, 387 (1998).
14. H. T. Orr, et al., Nature Genet. 4, 221 (1993); E. N. Burright, et al., Cell 82, 937-948 (1995); P. J. Skinner, et al., Nature 389, 971 (1997); H. B. Clark, et al., J. Neurosci. 17, 7385 (1997)
15. S. M. Pulst, et al., Nature Genet. 14, 269 (1996).
16. Y. Kawaguchi, et al., ibid. 8, 221 (1994); H. Ikeda et al., ibid. 13, 196 (1996); H. L. Paulson, et al., Neuron 19, 333 (1997).
17. K. Flanigan, et al., Am. J. Hum. Genet. 59, 392 (1996).
18. L. P. Ranum, et al., Nature Genet. 8, 280 (1994).
19. O. Zhuchenko, et al., Nature Genet. 15, 62 (1997).
20. G. David, et al., Nature Genet. 17, 65 (1997); M. Holberg, et al., Hum. Mol. Genet. 7, 913 (1998).

### Equivalents

Those skilled in the art will recognize, or be able to ascertain using no more than routine experimentation, many equivalents to the specific embodiments of the invention described herein. Such equivalents are intended to be encompassed by the following claims.

1. A method for inhibiting the formation of intracellular aggregates of selected polypeptides comprising, the step of contacting said polypeptide capable of forming said aggregates with a polypeptide-binding molecule that specifically binds to said polypeptide in a manner to minimize aggregation, thereby inhibiting the formation of said intracellular aggregates.

2. The method of claim 1, wherein said polypeptide is selected from the group consisting of Amyloid Precursor Protein, Presenilin 1, Presenilin 2, −2 Macroglobulin, Apolipoprotein, -Synuclein, huntingtin, Prion protein, Tau, SOD, AR, Atrophin 1, Ataxin 1, Ataxin 2, Ataxin 3, CACNL1A4, and SCA7.

3-6. (canceled)

7. The method of claim 1, wherein said polypeptide-binding molecule is selected from the group consisting of small molecules, peptides, peptidomimetics, antibodies, antibody fragments, and intrabodies.

8-35. (canceled)

39. A method for treating a subject having, or likely to have, a neurological disorder comprising, administering to said subject an polypeptide-binding molecule which specifically binds a polypeptide capable of forming a polypeptide aggregate associated with a neurological disorder thereby inhibiting said aggregate from forming.

40. The method of claim 39, wherein said neurological disorder is selected from the group consisting of Alzheimer's disease, Parkinson's disease, Huntington's disease, Prion disease, FTD, ALS, SBMA, DRPLA, SCA1, SCA2, SCA3/MJD, SCA4, SCA5, SCA6, and SCA7.

41-43. (canceled)

44. The method of claim 39, wherein said polypeptide is selected from the group consisting of Amyloid Precursor Protein, Presenilin 1, Presenilin 2, −2 Macroglobulin, Apolipoprotein, -Synuclein, huntingtin, Tau, SOD, AR, Atrophin 1, Ataxin 1, Ataxin 2, Ataxin 3, CACNL1A4, and SCA7.

45-48. (canceled)

49. The method of claim 39, wherein said polypeptide-binding molecule is selected from the group consisting small molecule, peptide, peptidomimetic, antibody, antibody fragment, and intrabody.

50-52. (canceled)

53. A method for identifying an polypeptide-binding molecule or a functional fragment thereof which specifically recognizes a polypeptide capable of forming intracellular polypeptide aggregates comprising,
providing a polypeptide capable of forming intracellular polypeptide aggregates;
contacting said polypeptide with a test polypeptide-binding molecule intrabody or functional fragment thereof; and
determining the ability of said test polypeptide-binding molecule intrabody or functional fragment thereof to specifically recognize said polypeptide,
thereby identifying an polypeptide-binding molecule which specifically recognizes a polypeptide capable of forming intracellular polypeptide aggregates.

54-55. (canceled)

56. The method of claim 53, wherein said polypeptide is selected from the group consisting of Amyloid Precursor Protein, Presenilin 1, Presenilin 2, −2 Macroglobulin, Apolipoprotein, -Synuclein, huntingtin, Tau, SOD, AR, Atrophin 1, Ataxin 1, Ataxin 2, Ataxin 3, CACNL1A4, and SCA7.

57-58. (canceled)

59. A method for identifying a compound which specifically recognizes a polypeptide capable of forming undesired intracellular polypeptide aggregates comprising,
providing a polypeptide capable of forming intracellular polypeptide aggregates;
providing a test intrabody or functional fragment thereof that binds said polypeptide;
incubating said polypeptide and intrabody fragment or fragment thereof with a binding molecule; and
determining the ability of said test compound to alter the binding of said intrabody or functional fragment thereof, wherein those binding molecules that bind the intrabody are eliminated,
thereby identifying said test compound as capable of interacting with a polypeptide capable of forming intracellular polypeptide aggregates.

60-62. (canceled)

63. The method of claim 59, wherein said polypeptide is selected from the group consisting of Amyloid Precursor Protein, Presenilin 1, Presenilin 2, −2 Macroglobulin, Apolipoprotein, -Synuclein, huntingtin, Tau, SOD, AR, Atrophin 1, Ataxin 1, Ataxin 2, Ataxin 3, CACNL1A4, and SCA7.

64-67. (canceled)

68. An isolated nucleic acid molecule encoding an intrabody, or functional fragment thereof, which binds to a selected polypeptide capable of forming intracellular polypeptide aggregates associated with a neurological disorder.

69. The molecule of claim 68, wherein said polypeptide is selected from the group consisting of Amyloid Precursor Protein, Presenilin 1, Presenilin 2, −2 Macroglobulin, Apolipoprotein, -Synuclein, huntingtin, Prion protein, Tau, SOD, AR, Atrophin 1, Ataxin 1, Ataxin 2, Ataxin 3, CACNL1A4, and SCA7.

70-71. (canceled)

72. An intrabody, or functional fragment thereof, which binds to a selected polypeptide capable of forming intracellular polypeptide aggregates associated with a neurological disorder.

73. The molecule of claim 72, wherein said polypeptide is selected from the group consisting of Amyloid Precursor Protein, Presenilin 1, Presenilin 2, −2 Macroglobulin, Apolipoprotein, -Synuclein, huntingtin, Prion protein, Tau, SOD, AR, Atrophin 1, Ataxin 1, Ataxin 2, Ataxin 3, CACNL1A4, and SCA7.

74-77. (canceled)

78. A method for inhibiting the formation of intracellular aggregates of a selected polypeptide in animal comprising,
immunizing said animal with an immunogen having an epitope in common with said selected polypeptide, wherein said immunizing provokes a host antibody immune response sufficient for inhibiting the formation of intracellular aggregates of said selected polypeptide to occur.

79. (canceled)

80. The method of claim 78, wherein said polypeptide is selected from the group consisting of Amyloid Precursor Protein, Presenilin 1, Presenilin 2, −2 Macroglobulin, Apolipoprotein, -Synuclein, huntingtin, Prion protein, Tau, SOD, AR, Atrophin 1, Ataxin 1, Ataxin 2, Ataxin 3, CACNL1A4, and SCA7.

81-84. (canceled)

85. The method of claim 78, wherein said immunogen is a polypeptide comprising an epitope in common with a polypeptide selected from the group consisting of Amyloid Precursor Protein, Presenilin 1, Presenilin 2, −2 Macroglobulin, Apolipoprotein, -Synuclein, huntingtin, Prion protein, Tau, SOD, AR, Atrophin 1, Ataxin 1, Ataxin 2, Ataxin 3, CACNL1A4, and SCA7.

86-93. (canceled)


SEQUENCE LISTING (45 sequences)

SEQ ID NO: 1 - DNA, 345 bp, Artificial Sequence
  Description of Artificial Sequence Synthetic construct
caggtgcagctgcaggagtcggggggaggcttggtacagcctggggggtccctgagactctcctgtgcagcctctggattcaccttcagtagttatagcatgagctgggtccgccaggctccaggcaaggggctggagtgggtggcagttatatcatatgatggaagcaataaatactacgcagactccgtgaagggccgattcaccatctccagagacaattccaagaacacgctgtatcttcaaatgaacagcctgagagccgaggacacggccgtgtattactgtgcgagagataggtacttcgatctctggggccgtggcaccctggtcaccgtctcctca

SEQ ID NO: 2 - protein, 115 aa, Artificial Sequence
  Description of Artificial Sequence Synthetic construct
QVQLQESGGGLVQPGGSLRLSCAASGFTFSSYSMSWVRQAPGKGLEWVAVISYDGSNKYYADSVKGRFTISRDNSKNTLYLQMNSLRAEDTAVYYCARDRYFDLWGRGTLVTVSS

SEQ ID NO: 3 - DNA, 327 bp, Artificial Sequence
  Description of Artificial Sequence Synthetic construct
cagtctgccctgactcagcctgcctccgtgtctgggtctcctggacagtcgatcaccatctcctgcactggaaccagcagtgacattggtgcttataactatgtctcctggtaccagcagtatccaggcaaggcccccaaactccttatttatgatgtcagtaatcggccctcagggatttctaatcgcttctctggctccaagtctggcgatacggcctccctgaccatctctgggctccaggctgaggacgaggctgattattactgcagctcatttgcgaacagcggccccttattcggcggagggaccaaggtcaccgtccta

SEQ ID NO: 4 - protein, 109 aa, Artificial Sequence
  Description of Artificial Sequence Synthetic construct
QSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVL

SEQ ID NO: 5 - DNA, 717 bp, Artificial Sequence
  Description of Artificial Sequence Synthetic construct
caggtgcagctgcaggagtcggggggaggcttggtacagcctggggggtccctgagactctcctgtgcagcctctggattcaccttcagtagttatagcatgagctgggtccgccaggctccaggcaaggggctggagtgggtggcagttatatcatatgatggaagcaataaatactacgcagactccgtgaagggccgattcaccatctccagagacaattccaagaacacgctgtatcttcaaatgaacagcctgagagccgaggacacggccgtgtattactgtgcgagagataggtacttcgatctctggggccgtggcaccctggtcaccgtctcctcaggtggaggcggttcaggcggaggtggctctggcggtggcggatcgcagtctgccctgactcagcctgcctccgtgtctgggtctcctggacagtcgatcaccatctcctgcactggaaccagcagtgacattggtgcttataactatgtctcctggtaccagcagtatccaggcaaggcccccaaactccttatttatgatgtcagtaatcggccctcagggatttctaatcgcttctctggctccaagtctggcgatacggcctccctgaccatctctgggctccaggctgaggacgaggctgattattactgcagctcatttgcgaacagcggccccttattcggcggagggaccaaggtcaccgtccta

SEQ ID NO: 6 - protein, 239 aa, Artificial Sequence
  Description of Artificial Sequence Synthetic construct
QVQLQESGGGLVQPGGSLRLSCAASGFTFSSYSMSWVRQAPGKGLEWVAVISYDGSNKYYADSVKGRFTISRDNSKNTLYLQMNSLRAEDTAVYYCARDRYFDLWGRGTLVTVSSGGGGSGGGGSGGGGSQSALTQPASVSGSPGQSITISCTGTSSDIGAYNYVSWYQQYPGKAPKLLIYDVSNRPSGISNRFSGSKSGDTASLTISGLQAEDEADYYCSSFANSGPLFGGGTKVTVL

SEQ ID NO: 7 - DNA, 44 bp, Artificial Sequence
  Description of Artificial Sequence Synthetic construct
tcaccgtctcctcaggtggaggcggttcaggcggaggtggctct

SEQ ID NO: 8 - DNA, 48 bp, Artificial Sequence
  Description of Artificial Sequence Synthetic construct
tgggtgagctcatgtccgmtccgccaccgccagagccacctccgcctg

SEQ ID NO: 9 - protein, 69 aa, Artificial Sequence
  Description of Artificial Sequence Synthetic Construct
LVPRGSVSTHHHHHQQQQQQQQQQQQQQQQQQQQQQQQQQQQQQQQQQQHHGNSGPPEFPGRLERPHRD

SEQ ID NO: 10 - protein, 59 aa, Artificial Sequence
  Description of Artificial Sequence Synthetic Construct
LVPRGSMATLEKLMKAFESLKSFQQQQQQQQQQQQQQQQQQQQQQQQQLQPGSTRAAAS

SEQ ID NO: 11 - protein, 76 aa, Artificial Sequence
  Description of Artificial Sequence Synthetic Construct
LVPRGSMATLEKLMKAFESLKSFQQQQQQQQQQQQQQQQQQQQQQQQQQQQQQQQQQQQQQQQQQLQPGSTRAAAS

SEQ ID NO: 12 - protein, 64 aa, Artificial Sequence
  Description of Artificial Sequence Synthetic Construct
MATLEKLMKAFESLKSFQQQQQQQQQQQQQQQQQQQQQQQQQQQQQQQQQQQQQQQQQQQQQQQ

SEQ ID NO: 13 - protein, 89 aa, Artificial Sequence
  Description of Artificial Sequence Synthetic Construct
MATLEKLMKAFESLKSFQQQQQQQQQQQQQQQQQQQQQQQQQQQQQQQQQQQQQQQQQQQQQQQQQQQQQQQQQQQQQQQQQQQQQQQQ

SEQ ID NO: 14 - protein, 121 aa, Artificial Sequence
  Description of Artificial Sequence Synthetic Construct
MATLEKLMKAFESLKSFQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQ

SEQ ID NO: 15 - protein, 98 aa, Artificial Sequence
  Description of Artificial Sequence Synthetic Construct
MATLEKLMKAFESLKSFQQQQQQQQQQQQQQQQQQQQQQQQQQQQQQQQQQQQQQQQQQQQQQQLQPGGSTMSRGPFEQKLISEEDLNMHTEHHHHHH

SEQ ID NO: 16 - protein, 123 aa, Artificial Sequence
  Description of Artificial Sequence Synthetic Construct
MATLEKLMKAFESLKSFQQQQQQQQQQQQQQQQQQQQQQQQQQQQQQQQQQQQQQQQQQQQQQQQQQQQQQQQQQQQQQQQQQQQQQQQLQPGGSTMSRGPFEQKLISEEDLNMHTEHHHHHH

SEQ ID NO: 17 - protein, 155 aa, Artificial Sequence
  Description of Artificial Sequence Synthetic Construct
MATLEKLMKAFESLKSFQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQLQPGGSTMSRGPFEQKLISEEDLNMHTEHHHHHH

SEQ ID NO: 18 - protein, 66 aa, Artificial Sequence
  Description of Artificial Sequence Synthetic Construct
IDGGGGGKGPVTGTGSMATLEKLMKAFESLKSFQQQQQQQQQQQQQQQQQQQQQQQQQLQPRILTN

SEQ ID NO: 19 - protein, 145 aa, Artificial Sequence
  Description of Artificial Sequence Synthetic Construct
IDGGGGGKGPVTGTGSMATLEKLMKAFESLKSFQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQQLQPRILTN

SEQ ID NO: 20 - protein, 113 aa, Artificial Sequence
  Description of Artificial Sequence Synthetic Construct
IDGGGGGKGPVTGTGSVSTHHHHHQQQQQQQQQQQQQQQQQQQQQQQQQQQQQQQQQQQQQQQQQQQQQQQQQQQQQQQQQQQQQQQQQQQQQQQQQQQQQQQQQHHSGPPEF

SEQ ID NO: 21 - protein, 79 aa, Artificial Sequence
  Description of Artificial Sequence Synthetic construct
MATLEKLMKAFESLKSFQQQQQQQQQQQQQQQQQQQQQQQPPPPPPPPPPPQLPQPPPQAQPLLPQPQPPPPPPPPPPG

SEQ ID NO: 22 - protein, 70 aa, Artificial Sequence
  Description of Artificial Sequence Synthetic construct
HPEAASAAPPGASLLLLQQQQQQQQQQQQQQQQQQQQQETSPRQQQQQQGEDGSPQAHRRGPTGYLVLDE

SEQ ID NO: 23 - protein, 64 aa, Artificial Sequence
  Description of Artificial Sequence Synthetic construct
GAQETAHPPVATHHHHHQQQQQQQQQQQQQQHHGNSGPPPPGAFPHPLEGGSSHHAHPYAMSPS

SEQ ID NO: 24 - protein, 81 aa, Artificial Sequence
  Description of Artificial Sequence Synthetic construct
LLANMGSLSQTPGHKAEQQQQQQQQQQQQHQHQQQQQQQQQQQQQQQHLSRAPGLITPGSPPPAQQNGYVHISSSPQNTGR

SEQ ID NO: 25 - protein, 72 aa, Artificial Sequence
  Description of Artificial Sequence Synthetic construct
RPACEPVYGPLTMSLKPQQQQQQQQQQQQQQQQQQQQQQPPPAAANVRKPGGSGLLASPAAAPSPSSSSVSS

SEQ ID NO: 26 - protein, 72 aa, Artificial Sequence
  Description of Artificial Sequence Synthetic construct
EELRKRREAYFEKQQQKQQQQQQQQQQQQQQQQQQQQQQRDLSGQSSHPCERPATSSGALGSDLGKACSPFI

SEQ ID NO: 27 - protein, 80 aa, Artificial Sequence
  Description of Artificial Sequence Synthetic construct
QPIQNTNSLSILEEQQRQQQQQQQQQQQQQQQQQQQQQQQQQQQQQQQQQQQQQQAVAAAAVQQSTSQQATQGTSGQAPQ

SEQ ID NO: 28 - protein, 9 aa, Artificial Sequence
  Description of Artificial Sequence Synthetic construct
YPYDVPDYA

SEQ ID NO: 29 - protein, 15 aa, Artificial Sequence
  Description of Artificial Sequence Synthetic construct
YDVPDYATPPLLLLV

SEQ ID NO: 30 - protein, 25 aa, Artificial Sequence
  Description of Artificial Sequence Synthetic construct
YPYDVPDYASDKQTLLQNEQLYQPL

SEQ ID NO: 31 - protein, 27 aa, Artificial Sequence
  Description of Artificial Sequence Synthetic construct
YPYDVPDYASKDGKKKKKKSKTKCVIM

SEQ ID NO: 32 - protein, 15 aa, Artificial Sequence
  Description of Artificial Sequence Synthetic construct
YPYDVPDYASEKDEL

SEQ ID NO: 33 - protein, 12 aa, Artificial Sequence
  Description of Artificial Sequence Synthetic construct
YPYDVPDYASKL

SEQ ID NO: 34 - protein, 38 aa, Artificial Sequence
  Description of Artificial Sequence Synthetic construct
YPYDVPDYAHIKVRRKNIFEDAYQEIMRQTPEDLKKRL

SEQ ID NO: 35 - protein, 41 aa, Artificial Sequence
  Description of Artificial Sequence Synthetic construct
MIKFDGEEGLDYGGVSREFFFLLSHEMFNPFYCLFEYSAYD

SEQ ID NO: 36 - protein, 40 aa, Artificial Sequence
  Description of Artificial Sequence Synthetic construct
NYTIQINPNSGINPEHLNYFKFIGRVVGLGVFHRRFLDAF

SEQ ID NO: 37 - protein, 31 aa, Artificial Sequence
  Description of Artificial Sequence Synthetic construct
FVGALYKMMLRKKVVLQDMEGVDAEVYNSLN

SEQ ID NO: 38 - protein, 43 aa, Artificial Sequence
  Description of Artificial Sequence Synthetic construct
WMLENSIDGVLDLTFSADDERFGEVVTVDLKPDGRNIEVTDGN

SEQ ID NO: 39 - protein, 43 aa, Artificial Sequence
  Description of Artificial Sequence Synthetic construct
KKEYVELYTQWRIVDRVQEQFKAFMDGFNELIPEDLVTVFDER

SEQ ID NO: 40 - protein, 43 aa, Artificial Sequence
  Description of Artificial Sequence Synthetic construct
ELELLIGGIAEIDIEDWKKHTDYRGYQESDEVIQWFWKCVSEW

SEQ ID NO: 41 - protein, 44 aa, Artificial Sequence
  Description of Artificial Sequence Synthetic construct
DNEQRARLLQFTTGTSRIPVNGFKDLQGSDGPRRFTIEKAGEVQ

SEQ ID NO: 42 - protein, 40 aa, Artificial Sequence
  Description of Artificial Sequence Synthetic construct
QLPKSHTCFNRVDLPQYVDYDSMKQKLTLAVEETIGFGQE

SEQ ID NO: 43 - protein, 9 aa, Artificial Sequence
  Description of Artificial Sequence Synthetic construct
YPYDVPDYA

SEQ ID NO: 44 - protein, 9 aa, Artificial Sequence
  Description of Artificial Sequence Synthetic construct
TPPLLLRLV

SEQ ID NO: 45 - protein, 10 aa, Artificial Sequence
  Description of Artificial Sequence Synthetic construct
EQKLISEEDL